(19) United States
(12) Patent Application Publication
(10) Pub. No.: US 2012/0295977 A1
(43) Pub. Date: Nov. 22, 2012

(54) Organosulfur prodrugs for the prevention and treatment of infectious diseases

(76) Inventor: David M. Ott, Oakland, CA (US)

(21) Appl. No.: 13/507,307

(22) Filed: Jun. 20, 2012

Related U.S. Application Data
(63) Continuation-in-part of application No. 10/853,415, filed on May 24, 2004, now Pat. No. 8,222,299.
(60) Provisional application No. 60/472,791, filed on May 23, 2003.

(51) Int. Cl. A61K 31/095; A61P 11/00; A61P 29/00; A61K 31/198; A61P 31/00

(52) U.S. Cl. 514/562; 514/706

(57) ABSTRACT

A method for enhancing the overall beneficial immune system response in a host that works in conjunction with the host's natural immune system response to enhance the host's ability to eliminate infectious microbes while simultaneously suppressing the toxicity of the immune system response to the host. Allium related organosulfur compounds are disclosed which have a various antimicrobial immunomodulatory properties that work together with the host's immune system in the prevention and treatment of disease. Prophylactic and therapeutic treatment is provided by administering an allium related organosulfur compound such that a localized thiosulfinate is caused to be non-enzymatically formed in response to localized generation by activated immune system cells of reactive oxygen species such as hydrogen peroxide. Suitable allium related organosulfur compounds may be administered to the host in an efficient manner through the use of S-AllylMercapto-N-AcetylCysteine (SAMNAC) or similar prodrugs.

This application is a continuation in part of application Ser. No. 10/853,415 which was filed on May 24, 2004.

### 1. BACKGROUND OF THE INVENTION

### 1.1 Field of the Invention

The present invention applies generally to the prevention and treatment of infectious diseases and/or pathologenic immune system response, and is more specifically illustrated in the prevention and treatment of bacterial infections, pneumonia, and Acute Respiratory Distress Syndrome (ARDS).

### 2. DEFINITIONS, ORGANOSULFUR GLOSSARY, AND ABBREVIATIONS

### 2.1 Definitions and Organosulfur Glossary

Allicin: Chemical name DiAllylThioSulfinate; chemical formula:

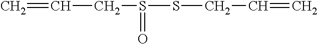

A compound formed by crushing garlic (which allows the enzymatic conversion by alliinase of alliin to allicin) that produces many of the medicinal benefits that are attributed to garlic.

Allium related compounds: Organosulfur compounds that are either derived from alliums (e.g. garlic or onion) through chemical or metabolic means, or are related to such compounds in specific ways such that they can reasonably be expected to exhibit similar medicinal properties.

Allyl mercaptan: AllylSH, chemical name AllylThiol; chemical formula:

CH₂═CH—CH₂—SH

The primary pre-hepatic metabolite of allicin. In the presence of glutathione, two allyl mercaptan molecules are produced from each allicin molecule. This reaction is known to occur very rapidly within red blood cells.

Allyl mercapto radical: AllylS*, allyl mercaptan without the terminal hydrogen atom of the SH group, resulting in an unpaired electron on the sulfur atom which is available for bonding to the remainder of a larger molecule.

CH₂═CH—CH₂—S*

Cysteine: CySH, A sulfur-containing amino acid with the formula:

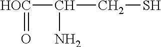

Cysteine is not to be confused with cysteine disulfide (CyS-SCy, generally referred to as cystine).

Cysteinal radical: CyS*, cysteine without the terminal hydrogen atom of the SH group, resulting in an unpaired electron on the sulfur atom. A cysteinal radical will typically be covalently bonded to another atom that is part of the complete molecule (e.g. with the sulfur atom of an allyl mercapto radical to form an S-AllylMercaptoCysteine molecule).

DiAllylDisulfide: DADS, (also abbreviated as AllylS-SAllyl), the disulfide formed from two AllylMercapto radicals bonded together. Equivalent to deoxygenated allicin.

CH₂═CH—CH₂—S—S—CH₂—CH═CH₂

Glutathione: GSH, the most omnipresent biothiol, a tripeptide composed of the amino acids glutamate, cysteine, and glycine. The symbol GSH uses “G” to represent the bulk of the molecule and “SH” to represent the thiol portion of the molecule, leading to symbols such as GS— for the ionized form of glutathione, GS* for the free radical form, and GSSG for the “oxidized” disulfide form.

Inflammatory Respiratory Distress:

Acute Respiratory Distress Syndrome (ARDS) is prototypical of conditions involving intense immune system responses that can be pathologenic. ARDS-like diseases that are addressed by the present invention include Acute Lung Injury (ALI), Systemic Inflammatory Response Syndrome (SIRS), Sepsis, Shock, Multiple Organ Dysfunction Syndrome (MODS), Compensatory Anti-inflammatory Response Syndrome (CARS), Mixed Antagonists Response Syndrome (MARS), and others. These diseases and conditions are jointly termed “inflammatory respiratory distress”.

Oxidized: The reaction product that tends to be produced when the reactants are exposed to oxygen, such as the conversion of thiols to disulfides. For example, if two cysteine molecules are exposed to reactive oxygen, their terminal “SH” hydrogen atoms tend to eventually disassociate from the molecules (e.g. they form thiyl radicals) and join together to form cysteine disulfide (also known as cystine). The two “abstracted” hydrogen atoms have combined with the reactive oxygen to form an H₂O molecule.

Oxidation tends to remove electrons from molecules. The removal of a hydrogen atom from a molecule is also considered to be oxidation, because this also involves the removal of an electron. Conversely, the removal of a proton (H⁺) is not considered oxidation, because the electron is left on the molecule.

Oxygenated: Another form of oxidation product, where an oxygen atom has been added to a molecule.

Pathologenic: Causing pathology.

Propyl mercaptan: Another mercaptan that is a natural metabolite from foods.

### Chemical Formula:

CH₃—CH₂—CH₂—SH

1-Propenyl mercaptan: Another mercaptan that is a natural metabolite from foods.

### Chemical Formula:

CH₃—CH═CH—SH

Reactive Oxygen Species: ROS, oxygen-containing molecules that are capable of producing oxidative damage to other molecules. Many, but not all, ROS are free radicals. Examples include (ISBN0306457563):
H₂O₂ (hydrogen peroxide), *O₂⁻ (superoxide radical), *OH (hydroxyl radical), HOCl (hypoclorus acid), ONOO⁻ (peroxynitrite), O₂¹ (singlet oxygen), O₃ (ozone) *NO (nitric oxide), and *NO₂ (nitrogen dioxide).

Reduced: The converse of oxidized. For example, when the terminal sulfur of a cystienal radical is bonded to a hydrogen atom to form a cysteine molecule, the cysteine is in its reduced state.

SAMC: S-AllylMercaptoCysteine, the molecule formed by a Cysteinal radical disulfide bonded to an AllylMercapto radical.

### Chemical Formula:

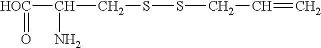

SAMG: S-AllylMercaptoGlutathione (also abbreviated as AllylS˜SG), the molecule formed by a glutathione radical disulfide bonded to an AllylMercapto radical.

SAMANC: S-AllylMercapto-N-Acetylcysteine. The molecule formed by an N-AcetylCysteinal radical disulfide bonded to an AllylMercapto radical.

### Chemical Formula:

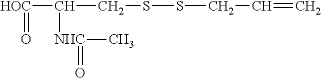

Thiol: Any molecule that includes one or more terminal sulfhydrate (SH) groups.

Thiyl radical: Multiple covalently bonded atoms that are considered to be a group terminating with a sulfur atom that has an unpaired electron. Normally, a thiyl radical will be covalently bonded to another atom that is part of the complete molecule, although it could alternatively be an unbonded “free radical.” For example, two cysteinal radicals with a covalent bond between their sulfur atoms (a disulfide bond) form a cysteine disulfide molecule (CyS-SCy, cystine).

### 2.2 Other Abbreviations

ARDS: Acute Respiratory Distress Syndrome

### 3. REFERENCES

For articles contained in books, the first reference is the book and the following reference(s) are the article(s).
A139:333; V. Dirsch et al; Effect of Allicin and Ajoene, two Compounds of Garlic, on Inducible Nitric Oxide Synthase; Atherosclerosis 139:333.
AA25:182; P. Mayeux et al; The Pharmacological effects of allicin, a constituent of garlic oil; Agents and Actions 25:182.
ABC52:2383; M. Tada et al; Nematicidal and Antimicrobial Constituents from Allium grayi Regel and Allium fistulosum L. var. caespiitosum; Agricultural and Biological Chemistry 52:2383.
AIT18:189; P. Josling; Preventing the Common Cold With a Garlic Supplement: A Double-Blind, Placebo-Controlled Study; Advances In Therapy 18:189.
AJCN74:756; M. Buono et at; Total Sulfur Amino Acid Requirement in Young Men as Determined by Indicator Amino Acid Oxidation; American Journal of Clinical Nutrition 74:756.
AJPLCMP265:L501; J. A. Leff et al; Postinsult treatment with N-acetyl-L-cysteine decreases IL-1-induced neutrophil influx and lung leak in rats; American Journal of Physiology Lung, Cell, and Molecular Physiology 265:L501.
AJPLCMP278:L1240; P. Knight et al; Acid Aspiration Increases Sensitivity to Increased Ambient Oxygen Concentrations; American Journal of Physiological Cell and Molecular Physiology 278, page L1240.
AM17:903; M. G. Johnson and R. H. Vaughn; Death of Salmonella typhimurium and Escherichia coli in the Presence of Freshly Reconstituted Dehydrated Garlic and Onion; Applied Microbiology 17:903.
ARB52:711; A. M. Meister, M. E. Anderson; Glutathione; Annual Review of Biochemistry 52:711.
ARZN28:250; J. Iravani et al; N-Acetylcysteine and Microciliary Activity in Mammalian Airways; Arzneimittel-Forschung 28:250.
BBA1379:233; A. Rabinkov, T. Miron, L. Konstantinovski, M. Wilchek, D. Mirelman, L. Weiner, 1998; The mode of action of allicin: trapping of radicals and interaction with thiol containing proteins; Biochimica et Biophysica Acta 1379:233.
BBA1463:20; T. Miron, A. Rabinkov, D. Mirelman, M. Wilchek, L. Weiner, 2000; The mode of action of allicin: its ready permeability through phospholipid membranes may contribute to its biological activity. Biochimica et Biophysica Acta 1463:20.
BJ63:514; E. Wills; Enzyme Inhibition by Allicin, the Active Principle of Garlic; The Biochemical Journal 63:514.
BMCCD2:2; M. Hayden and S. Tyagi; Is Type 2 Diabetes Mellitus a Vascular Disease (Atheroscleropathy) with hyperglycemia a late manifestation? The role of NOS, NO, and Redox Stress; BioMed Central Cardiovascular Diabetology 2:2.
BST23:S136; I. Das et al; Nitric Oxide Synthase is a Unique Mechanism of Garlic Action; Biochemical Society Transactions 23:S136.
C108:1315; G. U. Meduri et al; Plasma and BAL Cytokine Response to Corticosteroid Rescue Treatment in Late ARDS; Chest 108:1315.
C111:1157; Why Does Lactic Acidosis Occur in Acute Lung Injury?; Chest 111:1157.
C111:1301; J. Kellum et al; Release of Lactate by the Lung in Acute Lung Injury; Chest 111:1301.
C111:1306; A. S. Headley, E. Tolley, and G. U. Meduri; Infections and the Inflammatory Response in Acute Respiratory Distress Syndrome; Chest 111:1306.
C112:164; G. Bernard, et al; A Trial of Antioxidants N-acetylcysteine and Procysteine in ARDS; Chest 112:164
CIRCUL84:516; S. Kokura et al; Molecular Mechanisms of Neutrophil-Endothelial Cell Adhesion Induced by Redox Imbalance; Circulation Research 84:516.
COCC7:1; W. Lee and G. Downey; Neutrophil activation and Acute Lung Injury; Current Opinion in Critical Care 7:1.
CR61:725; H. Shirin et al; Antiproliferative Effects of S-Allylmercaptocysteine on Colon Cancer Cells When Tested Alone or in Combination with Sulindac Sulfide; Cancer Research 61:725.
DMD27:835; C. Teyssier et al; Metabolism of Dially Disulfide by Human Liver Microsomal Cytochromes P-450 and Flavin-Containing Monooxogenases; Drug Metabolism and Disposition 27:835.
EJP433:177; I. Sumioka, et al; Therapeutic effect of S-allylmercaptocysteine on acetaminophen-induced liver injury in mice; European Journal of Pharmacology 433:177.
FM29:348; K. N. Silashikanth, et al; A Comparative Study of Raw Garlic Extract and Tetracycline on Caecal Microflora and Serum Proteins of Albino Rats; Folia Microbiologica 29:348.
FRBM30:747; K. Kim et al; Differential Regulation of NO Availability from Macrophages and Endotheial Cells by the Garlic Component S-Allyl Cysteine; Free Radical Biology & Medicine 30:747.
GAS1953; selected pages of S. Colowick et al; Glutathione—A Symposium; Academic Press, New York, 1954.
ICM27:1699; I. Frerking et al; Pulmonary surfactant: functions, abnormalities and therapeutic options; Intensive Care Medicine 27:1699.
IJEB15:466; V. Sharma et al; Antibacterial Property of Allium SativumLinn.: in vivo & in vitro Studies; Indian Journal of Experimental Biology 15:466.
IJEB34:634; K. T. Agusti; Therapeutic values of onion (Allium cepa L.) and garlic (Allium sativum L.); Indian Journal of Experimental Biology 34:634.
ISBN0683181475; H. P. Koch and L. D. Lawson, 1996; GARLIC The Science and Therapeutic Application of Allium sativum L. and Relates Species; Williams & Wilkins, Baltimore Md.
ISBN0683181475:148; H. P. Koch and L. D. Lawson; Effects on Blood Pressure, Vascular Resistance, and Heart Function (in ISBN0683181475).
ISBN08176229416; Edited by C. Pasquier, R. Oliver, C. Auclair, and L. Packer, 1994; Oxidative Stress, Cell Activation and Viral Infection; Birkhauser Verlag, Boston, Mass.
ISBN08176229416:101; A. Meister; The Antioxidant Effects of Glutathione and Ascorbic Acid (in ISBN08176229416).
ISBN08176229416:143; G. Rotilio, L. Knoepfel, C. Steinkuhler, A. Palamara, M. Ciriolo, and E. Garaci; Effects of Intracellular Redox Stress on Cellular Regulation and Viral Infection (in ISBN08176229416).

ISBN1568360428; S, and E. Delany with A. Hearth, 1994; The Delany Sisters' Book of Everyday Wisdom; Kodansha International, New York.
ISBN1568360428:107; The Order of the Day (in ISBN1568360428).
ISBN1573312851; Edited by S. Park et al; Healthy Aging for Functional Longevity: Molecular and Cellular Interactions in Senescence; Annals of the New York Academy of Sciences, Volume 928.
ISBN1573312851:327; H. Chung et al; The Inflammation Hypothesis of Aging: Molecular Modulation by Caloric Restriction; (in ISBN1573312851).
ISBN9679786846; W. Fuchin and D. Yuhua 1998; Ginger, Garlic & Green Onion as Medicine; Pelanduk Publications, Malaysia.
JAFC44:3426; G. Cao et al; Antioxidant Capacity of Tea and Common Vegetables; Journal of Agricultural and Food Chemistry 44:3426.
JAFC49:2592; L. Lawson and Z. Wang, 2001; Low Allicin Release from Garlic Supplements: a Major Problem Due to the Sensitivities of Alliinase Activity; Journal of Agricultural and Food Chemistry 49:2592.
JAP89:353; A. Kaye et al; Analysis of Responses of Garlic Derivatives in the Pulmonary Vascular Bed of the Rat; Journal of Applied Physiology 89:353.
JBC259:399; S. T. Test et al; Quantitative and Temporal Characterization of the Extracellular H₂O₂ Pool Generated by Human Neutrophils; The Journal of Biological Chemistry 259:399.
JFCA2:327; G. Wierzbicka et al; Glutathione in Food; Journal of Food Composition and Analysis 2:327.
JI157:1630; T. S. Blackwell et al; In Vivo Antioxidant Treatment Suppresses Nuclear Factor-kB Activation and Neutrophilic Lung Inflammation; The Journal of Immunology 157:1630.
JID185:1115; P. Villa et al; Glutathione Protects Mice from Lethal Sepsis by Limiting Inflammation and Potentiating Host Defense; The Journal of Infectious Diseases 185:1115.
JN131:985 S; B. Lau; Suppression of LDL Oxidation by Garlic; The Journal of Nutrition 131:985 S, supplement 3S.
JN131:1010 S; C. Borek; Antioxidant Health Effects of Aged Garlic Extract; The Journal of Nutrition 131:1010 S, supplement 3S.
JN131:1041S; C. Yang et al; Mechanisms of Inhibition of Chemical Toxicity and Carcinogenesis by Diallyl Sulfide (DAS) and Related Compounds from Garlic; The Journal of Nutrition 131:1041S, supplement 3S.
JN131:1067S; D. Lamm and D. Riggs; Enhanced Immunocompetence by Garlic: Role in Bladder Cancer and Other Malignancies; The Journal of Nutrition 131:1067S, supplement 3S.
JN131:1109 S; T. Hoshino et al; Effects of Garlic Preparations on the Gastrointestinal Mucosa; The Journal of Nutrition 131:1109S, supplement 3S.
JSR65:165; S. L. Blair et al; Oral L-2-Oxo-4-thiazolidine Reduces Bacterila Translocation after Radiation in the Fischer Rat; Journal of Surgical Research 65:165.
MI2:125; S. Ankri, D. Mirelman; Antimicrobial Properties of Allicin from Garlic; Microbes and Infection 2:125.
N207:1269; R. Hamm and K Hoffman; Changes in the Sulphydryl and Disulfide Groups in Beef Muscle Proteins During Heating; Nature 207:1269.
NEJM344:699; G. Bernard et al; Efficacy and Safety of Recombinant Human Activated Protein C for Severe Sepsis; The New England Journal of Medicine 344:699.
PHRE59:527; B. Chance et al; Hydroperoxide Metabolism in Mammalian Organs; Physiological Reviews 59:527.
PM51:460; Y. Tsai et al; Antiviral Properties of Garlic: In vitro Effects on Influenza B, Herpes Simplex, and Coxsackie Viruses; Planta Medica 51:460.
PM53:305; H. Wagner et al; Effects of Garlic Constituents on Arachidonate Metabolism; Planta Medica 53:305.
PM58:417; N. Weber et al; In Vitro Virucidal Effects of Allium sativum (Garlic) Extracts and Compounds; Planta Medica 58:417.
PM59:A688; L. Lawson and Z. Wang, 1993; Pre-Hepatic Fate of the Organosulfur Compounds Derived from Garlic (Allium sativum); Planta Medica 59:A688.
PM60:417; J. Imai et al; Antioxidant and Radical Scavenging Effects of Aged Garlic Extract and its Constituents; Planta Medica 60:417.
PM67:13; L. Lawson, et al; Allicin Release under Simulated Gastrointestinal Conditions from Garlic Powder Tablets Employed in Clinical Trials on Serum Cholesterol; Planta Medica 67:13.
PMID12718222; I. V. Andrianova, et al; [Effect of long-acting garlic tablets “allicor” on the incidence of acute respiratory viral infections in children]; Ter Arkh. 20003; 75(3):53 [Article in Russian, PubMed abstract PMID 12718222].
PRR3:308; K. C. Meyer and J. J. Zimmerman; Inflammation and Surfactant; Pediatric Respiratory Reviews 3:308.
QK475.A43B56; E. Block; Garlic and Other Alliums—The Lore and the Science; RSC Publishing, Cambridge, UK.
QK475.A43B56:144; propyl vs. propenly groups from onion (in QK475.A43B56).
QP606.G59G59; N. Vermeulen et al, 1996; Glutathione S-Transfetases: Structure, Function and Clinical Implications; Taylor & Francis Ltd., London, England.
QP606.G59G59:199; T. Ishikawa and K. Akimaru; Transport of Glutathione S-Conjugates from Cancer Cells: Function and Structure of the GS-X Pump (in QP606.G59G59).
QRB73:3; J. Billing and P. W. Sherman; Antimicrobial Functions of Spices: Why Some Like it Hot; The Quarterly Review of Biology 73:3.
T55:46; T. Bauer et al; Comparison of systemic cytokine levels in patients with acute respiratory distress syndrome, severe pneumonia, and controls; Thorax 55:46.
TL69:15; R. White et al; Toxicity Evaluations of L-cysteine and Procysteine, a Cysteine Prodrug, Given Once Intravenously to Neonatal Rats; Toxicology Letters 69:15.
Torchinskii:1974; Y. Torchinskii, 1974; Sulfhydryl and Disulfide Groups of Proteins; Translated from Russion by H. Dixon, Consultants Bureau, New York.
U.S. Pat. No. 5,451,412A; G. Bounous et al; Biologically Active Undenatured Whey Protein Concentrate as Food Supplement; U.S. Pat. No. 5,451,412.
U.S. Pat. No. 7,678,833; David Ott; Method to Increase the Bioavailabity of Cysteine; U.S. Pat. No. 7,678,833.
US2005/0260250A1; David Ott; Medicinal Products Incorporating Bound Organosulfur Groups; USPTO Patent Application Publication US 2005/0260250A1.
U.S. Ser. No. 13/373,878; David Ott; Organosulfur Compounds for the Prevention and Treatment of Neurodegenerative Diseases; (patent application to be published).
VASP39:247; A. B. Johan Groeneveld; Vascular pharmacology of acute lung injury and acute respiratory distress syndrome; Vascular Pharmacology 39:247.
WO:01/36450; T. Miron et al; S-Allylmercaptoglutathione and Uses Thereof; World Intellectual Property Organization WO 01/36450 A1.

### 4. DESCRIPTION OF THE RELATED ART

### 4.1 Relationship with the Organosulfur Compounds from Alliums

Although the organosulfur compounds utilized by the present invention (e.g. protein-bound SAMC) are not necessarily constituents of alliums, they share a variety of properties with alliums. Hence known properties of alliums are germane to an appreciation of the invention and are reviewed here briefly.

4.1.1 Alliums and their Medicinal Benefits

Garlic, onions, and other alliums have been reputed to have beneficial medicinal properties for thousands of years. Traditional medicine has yielded a vast number of compositions containing garlic and/or onions for the treatment of a wide variety of conditions (ISBN9679786846, EPMR6:56, EPMR6:115).

Medicinal uses include the prevention and treatment of diseases related to the heart and circulatory system, microbial infections, cancer, respiratory diseases, hypoglycemia, and as an antidote for heavy metal poisoning and other toxins (ISBN0683181475, pages 135-211).

In the prevention of bacterial infections, garlic is exceptional as an antibiotic because it also produces a pro-biotic effect, encouraging the maintenance of a healthy intestinal flora (FM29:348). It simultaneously inhibits the “bad” bacteria (streptococci, coliforms, e. coli, salmonellae) by a large factor (100×) while inhibiting the “good” lactobacteria by a much smaller factor (10×). Another interesting aspect of garlic is the apparent inability of most bacteria to develop resistance to it (MI2:125), although apparently the lactic bacteria have evolved some tolerance.

In addition to disease prevention, garlic and other alliums have been used to provide general health benefits such as antioxidant protection, strengthened immune system, antihepitoxic protection, anti-inflammatory protection, improved digestion, and even for repelling insects (ISBN0683181475, pages 135-211).

Daily garlic consumption has been associated with health maintenance and is recommended for successful aging (ISBN1568360428:107). Literally thousands of scientific papers have been published on garlic, allicin, and related compounds, with 2240 references listed in ISBN0683181475, pages 235-319.

### 4.2 Allicin—an Effective Ingredient from Alliums, Especially Garlic

The first medicinal property of garlic to be studied with modern scientific methods was its antibacterial action (JACS66:1950). The active ingredient was isolated and given the name allicin.

The chemical structure of allicin was determined to be:

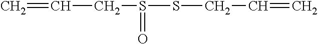

Numerous allium-related organosulfur compounds other than allicin have been found to provide medicinal benefits; however the benefits attributed to allicin tend to be the superset, in part because many of these other compounds produce similar metabolites in vivo (PM59:A688).

In the presence of glutathione and an active glutathione-reductase system, allicin is rapidly metabolized to allyl-mercaptan. This can be shown to occur in less than one minute by the analysis of blood cells that have been exposed to allicin (PM59:A688). The extremely high permeability of biological membranes to allicin also contributes to its biological activity (BBA1463:20).

Other garlic related organosulfur compounds that metabolize in blood to form allyl mercaptan include diallyl trisulfide, diallyl disulfide, ajoene, and SAMC (PM59:A688). It has been proposed that “in vitro or ex vivo studies of the mechanism of action of these compounds should not use the parent compounds, but rather should use allyl mercaptan, or possibly a further metabolite of allyl mercaptan” (ISBN0683181475, page 214).

4.2.1 Allicin is a Broad Spectrum Anti-Microbial Agent that is Effective in Preventing Infections, Including the Common Cold

Allicin has been shown to be a broad spectrum antimicrobial agent that significantly inhibits many strains of bacteria, fungi, parasites, and viruses (PM51:460, PM58:417, MI2:125). It has even been shown to be effective in the prevention and treatment of the common cold (AIT18:189), reducing the frequency of infection (24 vs 65 for the placebo group), and reducing the duration of symptoms to an average of 1.5 days (vs 5 days for the placebo group).

The mechanism of action was initially proposed to be due to allicin's reaction with cysteine (JACS66:1952), eliminating free SH groups (via disulfide formation) essential to bacterial proliferation. Allicin was subsequently shown to be a very potent inhibitor of “SH-enzymes” (BJ63:514). This model for its mechanism of action is still commonly accepted (MI2:125) and is supported by experimental evidence (BBA1379:233).

### 4.2.2 Allicin can be a Powerful Antioxidant, but not Always

Allicin typically has the general systemic effect of an antioxidant, but it also has some oxidant properties. It has been shown to scavenge H₂O₂ and *OH radicals in a concentration-dependent manner (MCB148:183). Although not observed by the authors of MCB148:183, their FIG. 7 shows that although at low to medium-high concentrations allicin is an antioxidant, at high concentrations t behaves as an oxidant resulting in the actual formation of *OH radicals.

In a study of the suppression of LDL oxidation by garlic related compounds (JN131:985S), S-allylcysteine, N-acetyl-S-allylcysteine, alliin, and especially SAMC were shown to significantly reduce Cu²⁺ induced LDL oxidation, but allicin increased the LDL oxidation to almost 3× the level of the control.

In a study of the total antioxidant capacity of 22 vegetables measuring the reduction of peroxyl radicals (*OOH), hydroxyl radicals (*OH), and of Cu²⁺ catalyzed free-radical chain reactions, garlic homogenate rated a “total antioxidant score” of 23.2 (second only to kale), approximately 3 times the average (JAFC44:3426). Interestingly, the garlic homogenate showed significant antioxidant capacity against Cu²⁺ induced oxidation (contrary to the effect of allicin reported in JN131:985 S).

In a chemiluminescense assay of the antioxidant properties of eight commercial garlic products, only the “AGE” product, (Aged Garlic Extract, from Wakugana Corporation, www.kyolic.com) had a net antioxidant activity (JN131:1010 S). The other products all contained garlic powder and produced allicin upon ingestion. The “AGE” product does not contain allicin but does contain primarily the water-soluble compounds S-allylcysteine (SAC) and SAMC, which are derived from allicin during the aging process.

In another study comparing the constituents of AGE with garlic extract, a chemiluminescense assay shows that AGE is an antioxidant but extracts from raw garlic or heated garlic are pro-oxidant (PM60:417). Of the water-soluble components of AGE, SAMC was shown to be by far the most effective antioxidant (by approximately a factor of two compared to the other compounds).

But Aged Garlic Extract also contains protein F4 from garlic, which is an immunostimulant that can cause inflammation (JN131:1067 S). In this case, the oxidants are produced in vivo by the immune system itself.

### 4.2.3 Toxicity of Allicin and Raw Garlic Powder

The comparative toxicity of different garlic preparations containing different garlic constituents has been studied (JN131:1109 S). Endoscopic examination of the stomach mucosa of dogs 24 hours after the direct administration of raw garlic powder detected erosion at 15 out of 18 sites. But if the garlic powder had been boiled (to inactivate the alliinase, thereby eliminating any allicin), no erosion was observed, but some redness appeared. When the “AGE” garlic product was administered (which contains no allicin), no erosion or redness was observed.

Enteric-coated garlic products release their contents (including enzymatically produced allicin) into the intestine (instead of the stomach). But examination of the intestine of a dog 3 hours after the administration of three tablets showed damaged and lost epithelial cells at the top of crypts (JN131:1109S). The authors questioned the safety of enteric-coated garlic products and recommended the use of AGE instead.

### 4.2.4 Sources of Allicin

### 4.2.4.1 Raw Garlic

When garlic is crushed, the enzyme alliinase converts the (previously separately compartmentalized) alliin instantly to allicin (ISBN0683181475, page 48). Interestingly, for the garlic plant itself this produces the antimicrobial agent precisely when and where it is needed, in response to the lysing of its cell walls by bacteria or fungi. (For humans it burns the mouth when chewed, probably due to oxidation.)

4.2.4.2 Garlic Supplements that Produce Allicin from Alliin

Dietary supplements traditionally do not directly incorporate allicin because of allicin's poor stability. Dietary supplements instead generally have contained the allicin precursor alliin along with the enzyme alliinase with an enteric coating utilized to prevent them from mixing together until they reach the intestine. The allicin release from these products has been problematic. If the coating dissolves too soon the stomach acids instantly deactivate the alliinase enzyme, but if the coating lasts too long the reaction never occurs. In a survey of dietary supplements published in 2001, only one supplement achieved its claimed bioavailable allicin yield (JAFC49:2592).

For example, in 1993 a change in the manufacturing process for “Kwai” garlic tablets caused their allicin yield to change from 73% of the theoretical yield to only 23%. This was discovered only after several clinical trials were conducted using these tablets (on the serum cholesterol lowering ability of garlic). In retrospect, the results of the various clinical trials can be seen to correlate with the actual allicin release from the various products tested (PM67:13).

### 4.2.4.3 Allicin Supplements Containing Pre-Formed Allicin

A process has been developed for stabilizing allicin, allowing the non-enzymatic delivery of allicin in a capsule. This product is currently only available from Allicin International (www.allimax.com). The actual allicin content is not printed on the label, nor is this information available from the manufacturer. Instead, the allicin content of each Allimax capsule is described as the same amount of allicin as you get from 1 clove of top quality garlic.

4.2.4.4 In vivo Allicin Production from DADS

Allicin is produced in the liver from Diallyl disulfide (DADS) via several cytochrome P-450 enzymes (e.g. CYP2E1) and flavin-containing monooxygenases (DMD27:835). Thus the in vivo production of allicin can be accomplished by any mechanism that delivers DADS to the liver. (Note: the DADS molecule is identical to an allicin molecule with the oxygen atom removed. Conversely, the oxygenation of a sulfur atom in a DADS molecule results in the formation of an allicin molecule.)

The activity of the enzymes is moderate (up to 121 pmol/min/pmol for CYP2D6), resulting in approximately 30% conversion of DADS to allicin in 30 minutes (DMD27:835).

The CYP2E1 enzyme can also oxygenate the allium-related compound diallyl sulfide to diallyl sulfoxide (DASO) and to diallyl sulfone (DASO2), which have been shown to inhibit carcinogenesis (JN131:1041S).

### 4.3 Cysteine—Amino Acid, Biothiol, and Glutathione Precursor

Cysteine is a sulfur containing amino acid which is an important constituent of proteins. The active site of many enzymes (e.g. proteases) involves cysteine, where the reactivity of the SH group contributes to the activity of the enzyme. Maintenance of the tertiary structure of proteins often depends on the formation of disulfide bonds between pairs of cysteines within the protein. Disulfide bonds can also link adjacent proteins, providing structure to tissues.

Cysteine is a thiol (it has a terminal “SH” group) and shares many properties with other thiols. It is able to participate in thiol-disulfide exchange reactions with almost all other thiols and disulfides resulting in a wide variety of mixed disulfides. Thiol-disulfide exchange reactions allow the formation of disulfide bonds (which are covalent bonds and quite strong) and their later separation with no energy involved (other than the thermal energy that brings the thiol and the disulfide together or apart).

Cysteine tends to auto-oxidize to cysteine disulfide (cystine) in the presence of oxygen. Inside cells the “reductive” environment provided by the maintenance of reduced glutathione (by glutathione reductase) tends to keep the cysteine reduced, but in an extracellular environment cysteine disulfide forms. Some types of cells can absorb cysteine disulfide, internally reduce it to cysteine, and then excrete the cysteine to the extracellular environment (AJM91_{—}3C:140S). This appears to be necessary for proper lymphocyte function and immune response.

Cysteine exhibits toxicity in large dosage, but non-toxic prodrugs exist (TL69:15), such as N-acetylcystine (NAC) and L-2-Oxo-thiazolidine (OTZ) (JSR65:165). The low solubility of cystine can result in the formation of kidney stones.

### 4.4 Glutathione—The Mother of all Antioxidants

Glutathione (GSH) serves as a critical antioxidant and is perhaps the only molecular antioxidant whose total depletion can directly cause death. In part, this is due to the ability of many antioxidants to “spare” for each other (even GSH can be partially spared by ascorbic acid). But it is also due to the ability of the glutathione reductase system to recycle almost all other antioxidants to their reduced state (ISBN08176229416:101). Therefore, insufficient GSH can result in the accumulated oxidation of various other antioxidants.

Glutathione's antioxidant properties are partially due to the various GSH-peroxidase enzymes that use GSH to reduce peroxides (e.g. hydrogen peroxide, H₂O₂), producing GSSG in the process, which in turn is reduced back to 2 GSH by GSH-reductase (ARB52:711). But glutathione can also react non-enzymatically to reduce H₂O₂, scavenge *O₂⁻ (superoxide) radicals, and detoxify reactive nitrogen (e.g. nitric-oxide) compounds.

Glutathione is also necessary for the detoxification of a wide variety of toxic substances (ARB52:711). The various GSH-transferase enzymes bind the toxic substances to glutathione molecules, which are then excreted from the cell (and ultimately from the body). Glutathione is a coenzyme for other detoxification processes, including the methylation of arsenic. Insufficient GSH (e.g. from depletion due to alcohol consumption) is responsible for acetaminophen (Tylenol®) toxicity, which is reported to be the second largest cause of toxic drug ingestion in the United States (BMCCC6:155).

### 4.5 Glutathione and/or Cysteine Deficiency

### 4.5.1 Dietary Sources Glutathione, Cysteine and Methionine

Cysteine deficiency may be common even in people who believe they eat enough protein. Many sources of dietary protein have low cysteine and methionine (another amino acid that can be converted to cysteine in vivo) content. People who do not eat much meat are especially at risk, especially the elderly. Even the official FAO/WHO/UNU recommendation for daily dietary cysteine+methionine consumption is reportedly low by almost a factor of two (13 mg/kg instead of 25 mg/kg) due to an arithmetic error when the requirements were determined experimentally in 1955 (AJCN74:756).

Glutathione in food varies dramatically such that well fed Americans are reported to exhibit a 40:1 range in its consumption (JFCA2:327). However, dietary glutathione probably has no special significance other than as a source of cysteine. The glutathione inside cells is created from its constituent amino acids (glutamate, cysteine, and glycine). Of these, cysteine is almost always the limiting amino acid.

A particularly good source of dietary cysteine is whey protein, which has been shown to increase glutathione levels, with a wide variety of associated health benefits (U.S. Pat. No. 5,451,412A, www.glutathone.com)

Dietary alliums are a good source of cysteine, but their unpleasant side effects when consumed in other than small quantities limit their ability to serve as a primary source of cysteine. But allium-related compounds (e.g. garlic) can cause an increase in the reduced glutathione level by increasing the activity of the GSH reductase enzyme by up to 87% (ISBN0683181475, page 190), thereby increasing the proportion of GSH to GSSG. Dietary garlic or onion powder has been shown to increase the liver glutathione level in chickens by 40% (ISBN0683181475, page 190). And exposure to SAMC has been shown to significantly increase the total glutathione level of cells (CR61:725).

4.5.2 Amino Acid Degradation from Food Processing and Cooking

While whey protein is an excellent source of cysteine, its bioavailable cysteine is very sensitive to denaturation from heat or mechanical shock, requiring a microfiltration process to be used during its manufacture (U.S. Pat. No. 5,451,412A). If not prevented, this denaturation causes a significant decrease in the ability of whey protein to raise the glutathione level (U.S. Pat. No. 5,451,412A).

The sulfhydryl and disulfide groups of proteins (i.e. the cysteine and cystine) are the most vulnerable amino acids to food processing and have been shown to be easily damaged by heat during cooking (N207:1269). Heating above 30 degrees C. causes progressive denaturation of, cystine, and heating above 70 degrees C. causes progressive irreversible destruction of cysteine (N207:1269).

### 4.5.3 Intense Delivery of Cysteine

Normally, the primary source of cysteine is dietary protein, but in some cases it is desirable to supply more cysteine than can reasonably be supplied through the consumption of protein. For example, the standard treatment for acetaminophen (Tylenol®) poisoning (which causes severe glutathione depletion) is the oral administration of N-acetylcysteine (NAC). Of necessity, the NAC dosage is high (an initial dose of 140 mg/kg, followed by 17 doses of 70 mg/kg every 4 hours). The low toxicity of NAC, combined with its rapid conversion to cysteine (which in turn is rapidly converted to glutathione inside liver cells) is important for this application.

Pretreatment with 100 mg/kg of SAMC has been shown to protect mice from acetaminophen poisoning, suppressing the reduction in hepatic glutathione level after acetaminophen administration (PHYRES3:50). In this study, SAC, which is the other major component Aged Garlic Extract (AGE) was not nearly as effective. Post treatment with a single dose of SAMC (200 mg/kg) shortly after exposure to acetaminophen is also protective in mice (EJP433:177).

SAMC administration has been shown to be a very efficient way to deliver cysteine directly into cells. In vitro experiments show that when cells are exposed to SAMC (which contains a cysteinal radical that is easily converted to cysteine), the result is a significant increase in the total glutathione level of cells, even beyond the level that glutathione is normally regulated to (CR61:725).

### 4.6 Thiol-Disulfide Exchange Reactions

Thiol-disulfide exchange reactions are a unique feature of organosulfur chemistry that provide a rapid, reversible, energy-neutral, highly specific covalent reaction for bonding together (or separating) molecules that incorporate thiol group or a disulfide bond (Torchinskii:1974).

More properly, this type of reaction should have been named the “thiolate-disulfide exchange reaction”, because it always involves the ionized version of the thiol. If the thiol is represented as RSH, and the disulfide as R′S˜SR″ the exchange is as follows:

RS⁻+R′S—SR″<->R′S⁻+RS—SR″ (or alternatively R″S⁻+R′S—SR)

In other words, the ion and the disulfide form a temporary complex with three inter-reacting sulfur atoms (and an electron), which soon separates with the resulting thiolate ion coming from any of the three thiyl radicals and the remaining disulfide molecule containing the other two thiyl radicals.

This brief description is necessarily simplified. Exchange reactions can be subject to steric constraints. And the products of the reaction depend on the relative redox potentials of the three thiyl radicals. But generally the reaction is rapid and the product mix is random, resulting in the formation of every possible mixed disulfide (and every possible thiol).

In practice, the reaction rate is pH dependent (due to the required ionization of the thiol). Also note that the total number of thiols is preserved (as is the total number of disulfide molecules). Only the mix has changed.

Thiol-disulfide reactions are important in the formation of the Cysteine-to-Cysteine bridges within proteins that help determine (and stabilize) the tertiary structure of the protein. They also are involved in the formation of Cysteine-to-Cysteine bridges between proteins.

Many enzymes have an “SH” group at their active site, and their activity depends on whether this remains an exposed thiol (or an exposed thiolate ion), with the enzyme being inactive if the thiol is “blocked” by an attached thiyl radical. This leads to the “redox regulation” of enzymes, which is an important mechanism for regulation, signaling, and control. Note that the inactivation of the enzyme is non-destructive, because a new thiol-disulfide exchange reaction between the blocked site and any thiolate ion that happens to float by can result in a disulfide floating away (leaving the SH group of the protein as a thiolate ion), and the enzyme becomes active again.

The majority of the organosulfur compounds that are discussed within this patent are thiols or disulfides, so these exchange reactions are highly relevant to their associated chemistry.

### 4.7 The Pre-Hepatic Fate of the Organosulfur Compounds Derived from Garlic

While the shear number of garlic-derived organosulfur compounds can present a confusing if not bewildering variety, nevertheless when they are consumed they all are exposed to a gastric environment (and therefore to dietary cysteine) and to blood (during transport from the intestine to the liver). An in vitro study was performed (PM59:A688) to determine the likely reaction products in these environments. The results are given in Table 1 of PM59:A688 and are summarized here (Table I) for the compounds most pertinent to the present invention.

TABLE I
Reactions of organosulfur compounds in the presence of blood or cysteine

| | Reaction with Cysteine | | Reaction in Blood | |
|---|---|---|---|---|
| | Half-life | Product | Half-life | Product |
| Compound | (min) | (moles) | (min) | (moles) |
| Allicin | <1 | SAMC (2) | <1 | AllylSH (1.6) |
| DADS | 45 | SAMC (1), | 60 | AllylSH (0.8) |
| | | AllylSH (1) | | |
| SAMC | NR | | 3 | AllylSH (0.8) |
| AllylSH | 80 | SAMC (0.8) | NR | |

These results show that regardless of the compound which is consumed, SAMC can be formed as an intermediate reaction product and AllylSH as the final product in blood. This has led to the recommendation that in vivo or in vitro studies on the mechanism of action of these compounds should not use the parent compound, but rather should use AllylSH or possibly a metabolite of AllylSH (ISBN0683181475, page 214).

### 4.8 The Prevention and Treatment of Bacterial Infection

In practice, the prevention of most bacterial infections is due to improved sanitary conditions. Immunization is also effective, but most people are not immunized against bacteria (beyond their childhood immunizations) unless there is a specific threat (e.g. immunization of military personnel against anthrax).

Experiments investigating methods for the prevention of bacterial infection induced by radiation treatment have shown that depletion of glutathione results in bacterial translocation (escape from the gut), and that OTZ treatment (which produces glutathione) is protective (JSR65:165).

The treatment of most bacterial infections is through the administration of antibiotics (or commonly, the management of the symptoms). The overuse (and misuse) of antibiotics for minor infections is a concern because this leads to the formation of resistant strains, which can result in untreatable, major infections. The use of antibiotics can also temporarily eliminate the “good” bacteria in the intestine, which can lead to superinfection because the good bacteria are no longer present to eliminate the “bad” bacteria.

Garlic and allicin have been extensively tested by researchers as antibiotics. In a review of antimicrobial spices, garlic inhibited all of the 30 types of bacteria that were tested (QRB73:3). This means that many people are actually consuming this antibiotic as part of their diet without really thinking about it.

In a comparison of the effectiveness of 13 types of antibiotics against 13 types of bacteria, Garlic and Chloramphenicol tied as the most effective antibiotics (inhibiting 12/13 of the species), and they also had the highest activity (average zone of inhibition of 20 mm) (IJEB15:466). Interestingly, the one type of bacteria that garlic was not effective against (Ps. Aeruginosa) was not inhibited by any of the other antibiotics either.

Rather than listing all of the bacteria that have been tested (and all of the associated references!), a summary of the more notable ones and the conditions that they can cause is presented:

### Causing Pneumonia:

Staphylococcus aureus, Strep. Pyogenes, Klebsiella pneumonae, Bacillus anthracis

### Causing Stomach or Urinary Tract Infections:

Salmonella typhimurium, E. coli, E. faecalis, E. durans

### Causing Ulcers and Stomach Cancer:

Helicobacter pylori

Causing Intestinal Gas after Legume Consumption:

Clostridium perfringens

While most of these antibacterial tests were in vitro, in vivo tests have been equally impressive. For example (IJEB15:466), when 4 week old chickens were fed 1 ml of crude garlic extract daily, the count of viable gram-negative bacilli per gram of rectal content was reduced by approximately a factor of 1000.

Apparently the “good” lactobacteria have evolved a tolerance to intestinal garlic (FM29:348), because while it inhibits the “bad” bacteria (streptococci, coliforms, e. coli, salmonellae) by a large factor (100×) in mice, it inhibits the “good” lactobacteria by a much lower amount (10×).

While not listed in detail here, garlic and allicin have also been shown to be antimicrobial against many fungi, viruses, parasites, etc. (MI2:125, ISBN0683181475), which are alternative forms of infection that are also subject to treatment within the scope of the present invention.

### 4.9 The Prevention and Treatment of ARDS

ARDS is normally treated by attempting to restore the lung's capacity for oxygen intake and by attempting to suppress the host's immune response. Most patients are of necessity treated after the development of ARDS (AJPLCMP265:L501). Mortality rates are on the order of 50% (C111:1306).

Mechanical ventilation (hyperbaric oxygen) produces mixed results, in part due to the formation of reactive oxygen species (ROS). The percentage of oxygen content that was used in the past was too high, especially given that the sensitivity to oxidative damage can be heightened in the conditions related to ARDS (e.g. acid aspiration (AJPLCMP278:L1240) or glutathione depletion).

Nitric Oxide (NO) (also administered via mechanical ventilation) produces mixed results, because although it both dilates blood vessels (good) it also can produce nitrogen-based ROS such as peroxinitrate (ONOO—) which are extremely damaging.

N-acetylcysteine (NAC) is a non-toxic prodrug that rapidly provides bioavailable cysteine and has produced beneficial results in treatment of ARDS in some tests (C112:164), inconclusive results in others, and may be detrimental at high dosages (VASP39:247). NAC is normally administered orally or intravenously, although occasionally intraparietal administration has been reported.

NAC has multiple beneficial effects, starting with the prevention of glutathione (GSH) depletion. Lowered levels of GSH increase neutrophil adhesion to endothelial cells (the first event in the lung that can lead to the development of ARDS). Interestingly, this is due to an increase in the adhesion molecules on the surface of endothelial cells, rather than a change in the neutrophils themselves (CIRCUL84:516).

Neutrophil influx into lung lavages is induced by interleukin 1 alpha (IL-1α), but this is inhibited by intravenous NAC administration (150 mg/kg), presumably due to GSH (or NAC itself) scavenging oxidants such as H₂O₂, HOCl, and *OH (AJPLCMP265:L501).

The generation of the cytokine-induced neutrophil chemoattractants which affect neutrophil migration is induced by NF-kB, which in turn is responsive the oxidative stress associated with GSH depletion. NAC treatment has been shown to decrease NF-kB activation, which in turn decreases neutrophilic inflammation in the lung (JI157:1630). Glutathione depletion also decreases the chemoattractive activity at the site of inflammation and can result in the improper migration of neutrophils to the lung in response to infections elsewhere (JID185:1115). Simultaneously, the decreased migration to the site of infection increases bacterial load and mortality. The antioxidant effect of NAC treatment markedly improves the survival of septic mice by simultaneously inhibiting inflammation while potentiating the host's innate immunity mechanisms.

NAC also increases mucus secretion, expectoration, and flow (in part due to its ability to increase cialary beat frequency (ARZN28:250), although high NAC concentrations (beyond 10⁻⁹ g/ml) suppress the cialary beat frequency and mucus flow (a reason not to use excessive NAC when treating ARDS).

OTZ (another prodrug that increases cysteine levels) has also been shown to be beneficial (C112:164), with the low toxicity of NAC (and OTZ) allowing high dosages to be used (e.g. over 10,000 mg/day for a 50 kg person).

Lung surfactant is comprised of phospholipids that reduce surface tension and greatly reduce the work of breathing. Surfactant replacement therapy, using either synthetic surfactant or calf's lung surfactant has shown some benefit in animal models, and shows promise in human trials PRR3:308), although it can exacerbate influenza infection (ICM27:1699). An increase in the surface tension of surfactant (which correlates with lowered concentrations surfactant-associated proteins) is an accurate early indicator of ARDS prognosis (AJRCCM160:1843). Those patients with high concentrations do not progress to ARDS, and those with low concentrations will have a high mortality rate. It has recently been shown that surfactant protein A (the most abundant one) is easily damaged by the oxidants such as nitrite (NO₂⁻), peroxynitrite (ONOO⁻, generated from *O₂⁻ and *NO), and hydrogen peroxide (H₂O₂), which are generated by activated neutrophils and other immune system cells during inflammation (FRBM33:1703).

Immune suppression through the use corticosteroid treatment also has had mixed results, but tends to be beneficial. Although the magnitude of the initial inflammatory response sets the pace for the evolution of ARDS, even patients with late onset ARDS and a low likelihood of survival can still benefit from the initiation of corticosteroid rescue treatment (C108:1315). But in many cases the response to corticosteroid rescue treatment is insufficient to protect the host and the excessive immune response remains more pathogenic than the infection. “The (poor) response to (corticosteroid rescue treatment) provides supporting evidence that ARDS patients most likely die with, rather than of, infection” (C111:1306).

Another form of treatment for ARDS that has been shown to give consistently beneficial results is supplementation with activated protein C, which has antithrombic, anti-inflammatory, and profibrinolytic effects (NEJM344:699).

It is interesting to note that garlic has also been shown to combine these effects (IJEB34:634). The amazing benefit of this was recently demonstrated in a study that utilized timed-release garlic powder tablets (Allicor, 300 mg/day). In a double-blind placebo controlled random 5-month trial in 42 children aged 10-12 years in comparison with 41 placebo-treated children, the incidence of acute respiratory viral infections in children was significantly reduced (PMID12718222). The morbidity was reduced 1.7-fold, and the “health index” was 1.5-fold higher compared to the placebo treated group. In an earlier study (using a dosage of 600 mg/day), 172 children aged 7-16 years were compared to 468 controls. In this study, the morbidity was reduced 2.4-fold as compared to the controls. Their conclusion is that allicor tablets are effective for non-specific prevention of acute respiratory infection in children and has no side effects. Note that the maximum allicin yield from the garlic powder in 600 mg of allicor tablets is 2.4 mg, so even the higher dosage was small compared to the dosages required by other means of treatment.

### 5. SUMMARY OF THE INVENTION

The present invention provides a method for enhancing the overall beneficial immune system response in a host that works in conjunction with the host's natural immune system response to simultaneously enhance the host's ability to eliminate infectious microbes while suppressing the pathologenic toxicity to the host of the host's immune system response.

The invention utilizes the non-enzymatic formation of allicin in response to the localized generation of H₂O₂ by immune system cells (such as neutrophils) to simultaneously increase the antimicrobial effect while reducing the cytotoxicity to the host. It is an advantage of the invention that it is able to nondestructively inhibit enzymes that would not normally be sensitive to deactivation by a thiol-disulfide exchange reaction. This results in part from the recognition that deactivation of SH dependent enzymes by allicin does not take place by the previously attributed mechanism of thiol-disulfide exchange reactions.

Briefly, in one of its aspects the invention calls for administering to a host an effective amount of an allium-related organosulfur compound such that a localized thiosulfinate is caused to be non-enzymatically formed in response to localized generation of H₂O₂ by the activated immune system cells.

It is shown that allicin, cysteine, and related organosulfur compounds have a variety of antimicrobial and immunomodulatory properties that work together with the host's immune system in the prevention and treatment of disease. The invention provides for simultaneous delivery of allicin, cysteine and related organosulfur compounds in an efficient manner, through the use of protein-bound S-AllylMercaptoCysteine, S-allylmercapto-N-acetylcysteine (SAMNAC), or similar prodrugs. The use of prodrugs avoids a variety of difficulties associated with the direct delivery of allicin or cysteine themselves.

A variety of illustrative modes of action are presented by which the invention may provide a beneficial combination of antimicrobial and anti-inflammatory modes of action.

The invention is also suitable for continuous preventative use in nutraceutical form, providing general health benefits while protecting from infectious diseases. Widespread use could provide “herd immunity”, increasing the protection of the general population.

The preferred embodiments are “low tech”, utilizing inexpensive ingredients and a simple manufacturing process, facilitating their widespread manufacture and use by economically disadvantaged groups.

The inventor has realized that there is a mechanism that can be utilized for the localized production of allicin in response to the host's localized generation of oxidants such as H₂O₂. It has been experimentally determined that the rate of allicin production from H₂O₂ is a non-linear function of the local H₂O₂ concentration, further enhancing the localization of the allicin production.

The inventor has also discovered that the mechanism of deactivation of many SH dependent enzymes by allicin is not by the previously attributed mechanism of thiol-disulfide exchange reactions, although in many ways the result is the same, and that allicin is able to nondestructively inhibit enzymes that would not be sensitive to deactivation by a thiol-disulfide exchange reaction.

The inventor has also discovered and developed new ways to formulate allium related compounds that have various advantages over existing dietary, dietary supplement, and medicinal products. These formulations may be utilized with the present invention.

Other aspects, advantages, and novel features of the invention are described below or will be readily apparent to those skilled in the art from the following specifications and drawings of illustrative embodiments.

### 6. BRIEF DESCRIPTION OF THE DRAWINGS

FIG. 1 shows the reduction of H₂O₂ by AllylSH, resulting in the non-enzymatic formation of diallyl-disulfide in the presence low H₂O₂ concentrations.

FIG. 2 shows the reduction of H₂O₂ by AllylSH and the further oxidation of diallyl-disulfide to allicin in the presence of high H₂O₂ concentrations.

### 7. DETAILED DESCRIPTION OF THE ILLUSTRATIVE EMBODIMENTS

The antimicrobial aspects of the invention are presented using various bacteria that can cause pneumonia as the prototypical infectious agents. Then the immunomodulatory aspects of the invention are presented, using Acute Respiratory Distress Syndrome (ARDS) as the prototypical pathogenic immune system response. A specific example is then introduced in which the various potential modes of action are illustrated with respect to the known characteristics of a single disease, Severe Acute Respiratory Distress Syndrome (SARS). Preliminary to these examples, a discussion of the localized production of allicin in the presence of the host's activated immune system cells is presented.

### 7.1 Localized Production of Allicin from AllylSH and/or DADS

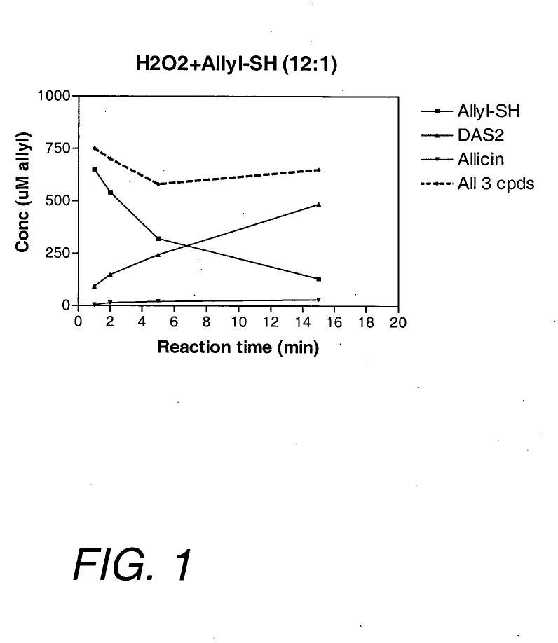

Thiols are known to have various antioxidant properties, including the ability to scavenge (H₂O₂), although the specific ability of allyl mercaptan in this regard may not have been previously investigated. It has been experimentally determined that allyl mercaptan (AllylSH) can reduce hydrogen peroxide (H₂O₂), oxidizing the AllylSH to diallyl disulfide (DADS, DAS2 in the figure) in the process (FIG. 1, initial concentrations of 10 mM H₂O₂ and 0.85 mM Allyl-SH).

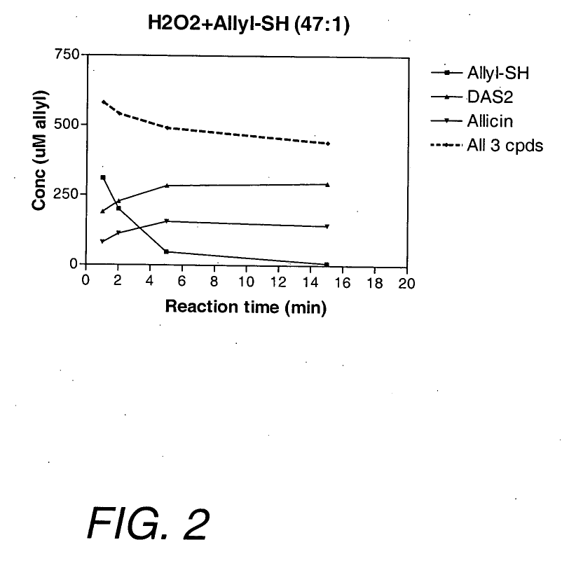

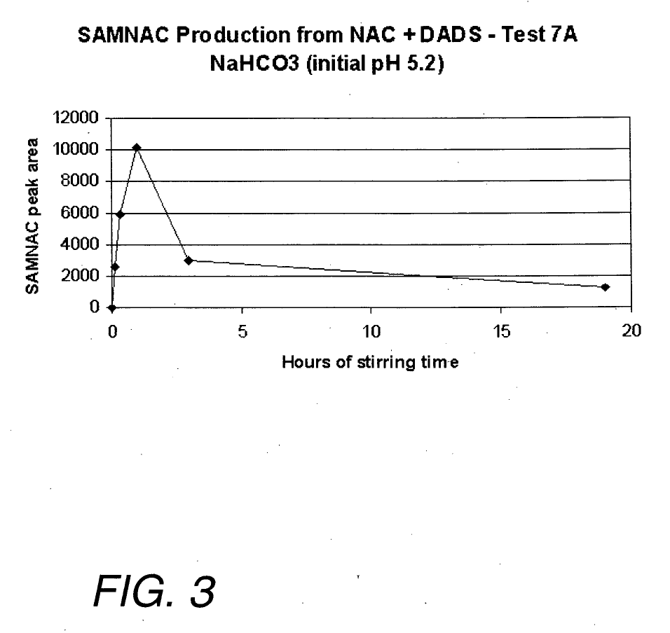

Surprisingly, at high concentrations of H₂O₂, the DADS was found to be further oxidized to Allicin (FIG. 2, initial concentrations 40 mM H₂O₂ and 0.85 mM AllylSH). Previously, allicin has only been known to form when the intermediate, sulfenic acid (AllylSOH) is first formed, as is the case when alliinase acts upon alliin.

For convenience, the relevant chemical formulas are repeated below:

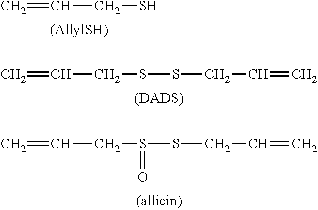

While not wanting to be bound to a particular theory, the following reaction mechanism is proposed for the production of DADS from AllylSH in the presence of H₂O₂:

AllylS⁻+H₂O₂->AllylSO⁻+H₂O

AllylSO⁻+AllylSH->DADS+OH⁻

While not wanting to be bound to a particular theory, the following reaction mechanism is proposed for the production of allicin from DADS in the presence of H₂O₂:

DADS+AllylSO⁻->allicin+AllylS⁻

According to this theory, because both the formation of DADS and the formation of allicin are dependent on the concentration of AllylSO⁻, which in turn is dependent on the concentration of H₂O₂, the resulting allicin concentration is a strong function of the H₂O₂ concentration. Therefore, any H₂O₂ concentration gradient produces a stronger gradient of allicin formation, and the localization of allicin formation is a strong function of the localization of H₂O₂ concentration.

The two H₂O₂ concentrations illustrated in FIGS. 1 and 2 are respectively representative of the environment non-local to an activated neutrophil (FIG. 1, 10 mM H₂O₂) and to the localized environment (within approximately 10 cell distances) adjacent to an activated neutrophil that is generating reactive oxygen species (ROS) (FIG. 2, 40 mM).

The neutrophil has an amazing ability to generate H₂O₂, as is indicated by experimental evidence (e.g. JBC259:399 “Quantitative and Temporal Characteristics of Extracellular H₂O₂ Pool Generated by Human Neutrophils”). Even when diluted in saline solution by approximately a factor of ten thousand (a diluted concentration of 3×10⁵/ml vs. an estimated packed cell density of 3×10⁹/ml), the H₂O₂ concentration produced by stimulated neutrophils reaches 12 uM. This corresponds to an undiluted H₂O₂ concentration of 120 mM. (Note that a dilution of a factor of ten thousand corresponds to a linear separation distance between the neutrophils of slightly over 20 cell diameters.)

The neutrophil is presented here as a prototypical type of cell that can generate extracellular ROS, but various other types of cells can also generate extracellular ROS (including not only cells of the immune system but also even some “normal” cells that can participate in an immune system response when activated). It will be appreciated that the various organosulfur compounds delivered to a host in accord with the invention may be effective in the environment of all such extracellular ROS generating cells, so that the invention may be applied beyond neutrophil cells alone.

### 7.1.1 Antioxidant Vs Pro-Oxidant Properties of Garlic and Allicin Explained.

Garlic and allicin have antioxidant properties because they are quickly metabolized to allyl mercaptan (allylSH), which has the same antioxidant properties as any thiol, including the ability to detoxify most forms of reactive oxygen and reactive nitrogen.

Garlic and allicin have pro-oxidant properties because the oxygen atom of the allicin molecule makes it somewhat unstable and capable of producing reactive oxygen through various reaction mechanisms. In particular, the reaction with cysteine produces as an intermediate product the sulfenic acid AllylSOH, which is so reactive that it is unlikely to be observed directly (see below).

The localized production of allicin from allylSH (via DADS) when exposed to a high concentration of H₂O₂ allows the antioxidant species to be distributed everywhere in the body and the pro-oxidant species only to be co-located with an attacking immune cell such as a neutrophil attacking an adjacent bacterium.

As will be shown below, this converts the non-specific cytotoxic agent H₂O₂ (generated by the neutrophil) to the more specific antimicrobial agent allicin.

In other words, by providing antioxidant protection from allylSH almost everywhere, and a more specific antimicrobial agent just where it is needed, the “selective killing index” of the natural immune system response is significantly increased beyond that of the innate immune system.

### 7.2 Enzyme Inactivation by Allicin is by a Thiol-Thiosulfinate Reaction

The rapid reaction of allicin with cysteine (<1 minute half-life) that was reported in PM59:A688 is in surprising contrast to the slow reaction of DADS with cysteine (45 minute half-life) that they also report. (See TABLE I in section 4.7 above.) If the reaction mechanisms both proceed by a thiol-disulfide exchange reaction, both reaction rates would be expected to be similar and to be rate-limited primarily by the concentration of CyS⁻ ions.

At a pH of 7, as an order of magnitude only 1/100 of the cysteine is ionized (the pKa of Cys is 8.53). It is interesting to note that the ratio of the half-life of allicin (say, 0.5 minutes) to the half-life of DADS is also approximately 1/100. This led the inventor to investigate whether the reaction with allicin could involve un-ionized cysteine directly, and therefore not be a thiol-disulfide exchange reaction. Because the concentration of un-ionized cysteine is approximately 100 times that of Cys⁻, this suggested an explanation for why allicin reacts so much faster than DADS.

Although the reaction of allicin with cysteine has been well established (and was first reported by Cavalitto in 1944 (JACS66:1952), no detailed investigation appears to have been performed prior to the investigation of the reaction of allicin with glutathione (which contains cysteine) by Miron et al (WO:01/36450). These results were very interesting.

First of all, they conclude that the forward reaction is:

2GSH+Allicin->2AllylS-SG+H₂O

They named AllylS˜SG “S-Allylmercaptoglutathione” because it consists of the mixed disulfide of allyl mercaptan and glutathione.

The present inventor's reasoned attempt at a reaction mechanism yielded:
1. Allicin+GSH->AllylS-SG+AllylSOH (oxygenated AllylSH)
2. AllylSOH+GSH->AllylS-SG+H₂O

Miron et al (WO:01/36450) also have shown that the reaction rate has a strong dependence on pH over the pH range of 5 to 7 and concluded that this indicates that the glutathione is in the form of a mercaptide ion (GS⁻). But the required participation of a mercaptide ion in the reaction is inconsistent with the hypothesis that the present inventor had formed that reaction with allicin could involve un-ionized cysteine directly. It was also inconsistent with the forward reaction mechanism advanced in the present disclosure.

The present inventor however has found a different explanation, not previously appreciated, for this pH dependence. Glutathione can convert to a thiazoline form (eliminating a water molecule in the process) at low pH (or even medium-low pH), and then it is no longer a thiol at all! (GAS1953, pages 21-29). At low pH, the infrared absorption spectrum clearly shows the absence of any cysteinal moiety and the establishment of the characteristic thiazoline peak at 2610 angstroms (GAS1953, page 24). The pKa of this transformation is 5.3 which, means that the transition between forms occurs gradually within the pH range of 5 to 7 (GAS1953, page 29), thus offering an alternative explanation of the pH dependence that is reported in WO:01/36450.

Confirmation of the feasibility of the reaction not involving any GS⁻ ions was eventually achieved when the present inventor found a description of the reaction of S⁻ monoxides (thiosulfinates) with thiols yielding mixed disulfides (Torchinskii:1974, page 95):

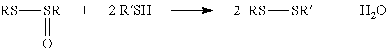

Substituting R=Allyl and R′SH=GSH yields the desired overall reaction.

Thus, the rapid reaction of allicin relative to diallyl disulfide has now been explained. It is a thiol-thiosulfinate reaction, not a thiol-disulfide reaction.

### 7.3 Example Benefits of the Invention

Many of the beneficial effects of the present invention are ultimately related either to the ability of the associated organosulfur compounds to form allicin, to participate in exchange reactions, to perform antioxidant functions, to inhibit (or activate) enzymes, or to enhance glutathione activity. These have been extensively described during the preceding sections. But the manifestations of these benefits are varied, as will be illustrated by examples of beneficial activity against a specific class of disease (pneumonia) and its potentially lethal complication (ARDS).

### 7.3.1 Benefits of the Invention for the Prevention and Treatment of ARDS

Acute Respiratory Distress Syndrome (ARDS) is prototypical of conditions involving intense immune system responses that can be pathological. ARDS-like diseases that are addressed by the present invention include Acute Lung Injury (ALI), Systemic Inflammatory Response Syndrome (SIRS), Sepsis, Shock, Multiple Organ Dysfunction Syndrome (MODS), Compensatory Anti-inflammatory Response Syndrome (CARS), Mixed Antagonists Response Syndrome (MARS), and others. These diseases and conditions are jointly termed “inflammatory respiratory distress”.

Many of the effects that have been attributed to allicin probably involve other intermediates in vivo. As has been discussed above, dietary allicin or digestive allicin formation does not directly result in systemic availability of allicin. The primary metabolite of allicin is allyl mercaptan, which is directly or indirectly responsible for various medicinal effects.

Because the dietary consumption of allicin or other compounds which contain allylmercapto groups (and metabolize to form allyl mercaptan) can locally produce allicin when the allyl mercaptan is exposed to oxidants in vivo, the broad spectrum antimicrobial activity of the allicin protects against a wide variety of microbes that can cause pneumonia, including both bacteria (such as Staphylococcus aureus, Strep. Pyogenes, Klebsiella pneumonae, Bacillus anthracis) and viruses (such as influenza, SARS, and the common cold).

Furthermore, the locally produced allicin can inhibit the replication of these microbes without permanently damaging host tissue, resolving the infectious disease without causing the lung injury which otherwise can be produced by an over-active immune system response.

### 7.3.1.1 Immunomodulatory Effects and Benefits

Neutrophils are representative of cells that can produce extracellular ROS in an attempt to damage nearby microbes. The following discussion is intended to apply in part to all other cell types with this ability, including other forms of polymorphonuclear leukocytes, mononuclear phagocytes, large granular lymphocytes, “killer cells”, and “normal” cells that can generate inflammatory extracellular ROS.

The initial onset of ARDS is extremely rapid, with significant damage to the lungs even in the first 15 minutes. The initial event seems to be the “sequestering” of neutrophils in small capillaries (some get stuck, causing a back up of even more) (COCC7:1). There is evidence that this in turn is initiated by intracellular inflammatory signaling molecules (cytokines) generated by the response to infection (C111:1306).

Enhancement of the endothelial NO Synthase enzyme (eNOS) is beneficial because its primary effect is circulatory dilation. But the enhancement of the inducible nitric oxide synthase enzyme (iNOS) in immune cells (such as neutrophils) is counterproductive in ARDS because its primary effect is the formation of ROS (BMCCD2:2). Therefore, inhibition of the iNOS is protective (A139:333). The administration of allicin (in this example and those below) has been shown to enhance eNOS activity while simultaneously inhibiting iNOS (BST23:S136). And various other garlic components also have this effect (FRBM30:747).

Another means of limiting inflammation is the inhibition of prostaglandin synthesis, which some allium related compounds have been shown to do amazingly well (diallyl disulfide at 50 uM produces 69% inhibition of prostaglandin synthase) (PM53:305).

ROS emitted by cells can cause nearby cells to also produce ROS, which can create a destructive feedback cycle (ISBN1573312851:327).

When ROS is generated by immune system cells to intentionally damage an adjacent infectious cell, the selective effectiveness of this process is dependent on the mean free path (lifetime) of the ROS until it either damages the intended victim, is scavenged by an antioxidant, or damages a host cell. Ideally, to avoid damage to other cells the mean free path would be on the order of one cell diameter. Therefore, in the presence of sufficient antioxidants, the likelihood of damaging a cell other than the intended one is low. But in the absence of antioxidants, the mean free path can extend to endanger many more nearby cells.

The antioxidant effects of compounds such as allyl mercaptan, other thiols, and even disulfides like diallyl disulfide (which now has been shown to reduce H₂O₂ when the H₂O₂ concentration is high) serve to limit the destructive range of ROS that diffuses away from its point of generation without significantly affecting the ability of neutrophils to damage microbes that are immediately adjacent.

Normally, the primary defense from H₂O₂ damage is provided by the enzymes catalase and glutathione peroxidase (PHRE59:527). But catalase saturates when the H₂O₂ concentration is high, so it provides limited protection adjacent to an active neutrophil. Glutathione peroxidase requires the involvement of glutathione and produces GSSG which in turn must be reduced back to GSH by glutathione reductase, so after some initial protection it is also likely to saturate. This means that the availability of nonenzymatic antioxidant protection may be more important than would otherwise be expected.

The ability of allyl mercaptan and diallyl disulfide to produce allicin in the presence of high concentrations of H₂O₂ converts this non-specific oxidant (hydrogen peroxide) which can produce a wide range of toxicities (and is implicated in the formation of a variety of more potent oxidants, such as the extremely reactive hydroxyl radical, *OH) into a much more specific and selective oxidant that is able to inhibit critical microbial enzymes, but has low toxicity to host cells.

The various properties attributed to NAC that are useful for the prevention and treatment of ARDS (see section 4.9 above) can also reasonably be expected to be properties of SAMC and especially of SAMNAC (see below). Most of the effects from NAC treatment relate to the ability of NAC to serve as a prodrug for cysteine or are attributed to the antioxidant properties of NAC due to its being a thiol. Like NAC, SAMC and SAMNAC produce cysteine in vivo, which is available for glutathione synthesis and as an antioxidant. SAMC and SAMNAC also produce allyl mercaptan, which provides an additional thiol antioxidant beyond that provided by NAC.

In diseases such as pneumonia from influenza, the damage to the lungs from the immune response of the host can exceed the damage from the infection itself. This is in common with potentially lethal viruses such as the SARS virus, where elevated systemic cytokine levels cause severe pneumonia (T55:46).

### 7.3.1.2 Physiological Effects and Benefits

Allicin enhances endothelial NO Synthase enzyme (eNOS) activity (see above), and the resulting NO causes dilation of blood vessels, which should reduce the likelihood of neutrophils becoming stuck in capillaries.

Allicin inhibits platelet aggregation, produces vasodilation, inhibits human neutrophil lysomal enzyme release, and promotes the maintenance of peripheral vasomotor tone (AA25:182). These effects may in turn be related to the inhibition of calcium uptake into platelets.

Allicin reduces platelet aggregation, which should also help keep the blood moving. Garlic has been shown to prevent hypoxic pulmonary hypertension in rats (AJP275: L283) which should be beneficial when ventilator assisted breathing is being used in ARDS treatment.

These effects attributed to garlic and allicin are likely to be due to their primary metabolite, allyl mercaptan.

A comprehensive summary of the effects of garlic and related compounds on blood pressure, vascular resistance, and heart function is contained in ISBN0683181475:148. The responses in rats are also detailed in (JAP89:353).

Lactic acidosis is characteristic of all forms of shock, including ARDS (C111:1157), and hyperlactemia is a sensitive indicator of the onset of ARDS (C111:1301). Allicin inhibits human neutrophil lysomal enzyme release (AA25:182), which may provide significant protection from this process.

### 7.4 Protein-Bound SAMC and Similar Prodrugs

Many of the properties of the relevant organosulfur compounds have been described above, but there are additional implementation-related considerations.

As explained above, suitable embodiments of organosulfur compounds must ultimately be able to form allicin, which means that they must be able to form DADS in vivo. In practice, this only requires that the selected compound include an allylmercapto group (AllylS) bound to the rest of the compound in a way that permits it to be freed during digestion. The simplest choices that meet this requirement are AllylSH itself, DADS, and allicin.

AllylSH has a stench that only a skunk could envy, which makes it commercially impractical in oral form. DADS has poor water solubility and has the taste and smell of garlic, again making it less commercially desirable. Allicin has some toxicity, which limits the potential dosage. It can give a burning sensation as it reacts with tissue. And it also tastes and smells like garlic.

Another consideration is that these compounds are now known to form AllylSSG when exposed to glutathione, which decreases the amount of free GSH in the cell. Due to the importance of glutathione, even a temporary depletion is undesirable, especially if a significant dosage is being used for the treatment of a disease in progress. This argues for the use of AllylSSG, because then as much glutathione is supplied as is being consumed.

But there are simpler and less expensive ways to administer a prodrug for AllylSH. AllylSSG provides little benefit over any other means of simultaneously delivering cysteine because GSH has been shown not to be transported into cells in its undigested form. (Even if it survived digestion, the cells would not absorb it.) So GSH (and by implication AllylSSG) is no more effective that any other source of dietary cysteine.

SAMC is a viable alternative to AllylSSG and is generally less expensive. Via thiol-disulfide exchange reactions, the SAMC molecules can form AllylSH and cysteine molecules during digestion.

But SAMC still has the taste and smell of garlic. This may not prove to be a commercial impediment since (for enteric coated tablets or capsules at least) the odor can be controlled by known methods.

In some sense the best (and least expensive) source of cysteine is dietary protein. This was a preferred composition for use at the time of the parent application Ser. No. 10/853,415 for which a notice of allowance was issued on May 3, 2012. A newer alternative is S-allylmercapto-N-acetylcysteine (“SAMNAC”, introduced in a co-pending application of the applicant, U.S. Ser. No. 13/373,878, filed on Dec. 5, 2011), which metabolizes to form both allyl mercaptan and N-acetylcysteine.

But the use of protein as the source of cysteine residues limits the reasonable dosage range when the composition is administrated in the form of tablets or capsules. The cysteine content of most types of protein is in the one percent range, so roughly speaking, a 1000 milligram capsule will only have 10 milligrams of cysteine, which can bind to only approximately 10 milligrams of allyl mercaptan.

Such a capsule is being manufactured by Viola Vitalis, Dhaka Bangladesh (a company partially owned by the applicant) for use in treatment of chronic arsenic poisoning (as described in U.S. Pat. No. 7,678,833, a patent of the applicant).

Here are some examples of newly preferred organosulfur compounds.

### 7.5 Synthesis of S-allylmercapto-N-acetylcysteine (SAMNAC)

Thiosulfinates react rapidly with mercaptans to form mixed disulfides (US2005/0260250A1). The thiosulfinate “allicin” reacts with a molecule of N-actylcysteine to form a molecule of SAMNAC plus a sulfenic acid that contains an allylmercapto group. This sulfenic acid molecule reacts rapidly with a second molecule of N-acetylcysteine to yield a second molecule of SAMNAC plus a molecule of water. These reactions between thiosulfinates and mercaptans are well known and are very rapid, and therefore were expected by the applicant to produce SAMNAC molecules with a high yield.

Synthesis of SAMNAC was performed for the'applicant as work for hire by Larry D. Lawson, Ph.D, Research Director of Silliker Inc. Utah Laboratory, using the following procedure:
1. Starting with 1.68 mg/ml of allicin in water, measure out 10.1 mL of allicin solution=16.9 mg=0.104 mmol allicin (this is 40% excess compared to 0.15 mmol N-acetylcysteine, as one molecule of allicin reacts with 2 molecules of N-acetylcysteine to form 2 molecules of SAMNAC).
2. To a 50-mL tube, add 24.5 mg N-acetylcysteine (0.15 mmol. M.W. 163).
3. Add the 10.1 mL allicin solution to the 24.5 mg of N-acetylcysteine.
4. Shake by hand until dissolved (20-40 sec).
5. Using 2N NaOH, adjust the pH from about 2.5 to about 4.1 (takes about 0.065 mL). The reaction will not proceed at pH 2.5 but is very fast at pH 4.1
6. Rotate for 30 min (reaction probably complete in the first 2-5 min).
7. Remove the excess allicin:
   to all or part of the reaction material in step 6, add 2 volumes of DCM
   shake by hand for 30 sec
   transfer the upper layer (SAMNAC solution) to another tube
Note: at the end of step 6, all of the N-acetylcysteine will have been used up, leaving only SAMNAC, excess allicin, some ajoene (allicin impurity), and the Na (from the NaOH, don't count the OH, as it is converted to water). Extraction with dichloromethane (DCM) removes >99% of the allicin and ajone and none of the SAMNAC, leaving only the SAMNAC, the added Na, and a trace of DCM.
8. The final amount & concentration of SAMNAC (M.W. 193) 10 mL of 15 millimolar (10 mg SAMNAC+3.0 mg added Na.
Note: The SAMNAC in solution is in the form of an anion (from the ionized carboxyl group). When the water is removed (by partial vacuum at 35 degrees C.), an oily yellow liquid id formed (which probably also contains a low concentration of the salt between the SAMNAC⁻ and the Na⁺ that was used to set the pH of the solution).

7.5.1 The Hydrogen NMR Spectrum Indicates that the SAMNAC is Authentic SAMNAC.

Dr. Jeff Conroy of Authentix Corporation confirmed that the NRM spectrum obtained by proton NMR from the dehydrated SAMNAC is consistent with the synthesized compound being authentic SAMNAC. The NMR spectrum is:

¹H NMR (CD₃OD, ppm): 1.99 (s, 3H), 2.99 (m, 1H), 3.31 (m, 1H), 3.36 (d, 2H), 4.44 (m, 1H), 5.091-5.21 (m, 2H), 5.79-5.93 (m, 1H).

7.5.2 Confirmation that SAMNAC Contains an Allyl Mercapto Group.

Analysis of SAMNAC to confirm that the compound contains an allyl mercapto group was performed as work for hire by Dr. Lawson. Starting with some of the SAMNAC produced above, treatment with the reducing agent tris(2-carboxyethyl)phosphate (TCEP) produces allyl mercaptan, as verified by HPLC.

7.5.3 Confirmation that SAMNAC Contains an N-acetylcysteinyl Group.

Analysis of SAMNAC to confirm that the compound contains an N-acetylcysteinyl group was performed as work for hire by Dr.Lawson. Starting with some of the SAMNAC produced above, treatment with the reducing agent tris(2-carboxyethyl)phosphate (TCEP) produces N-acetylcysteine, as verified by HPLC.

### 8 EXAMPLES OF COMPOSITIONS

Myself, some friends, and relatives have consumed various compositions which metabolize to form allyl mercaptan (or related mercaptans such as propyl mercaptan) for several years without any apparent negative effects.

I have experimentally consumed 240 mg/day of allyl mercaptan for several weeks without any apparent negative effects (except for taste and smell unpleasantness when taking each dose!). The results of a standard comprehensive blood test at the end of the experiment were essentially normal.

### 8.1 A “Garlic” Dietary Supplement Capsule

A dietary supplement capsule containing SAMNAC was produced by the following method:

1. Start with 5 bulbs of fresh garlic (330 g). This should produce approximately 1300 mg of allicin when pulverized, because the allicin yield from crushed garlic is approximately 4 mg/g (RM666.G15K6313:42).

2. Grate the garlic with a food processor (I used a KitchenAid food processor).

3. Transfer the “mash” to a blender (I used an Osterizer), add 200 mL of water, and blend for 10 minutes to completely pulverize the garlic.

4. Add 15 mL of N-acetylcysteine powder (11.5 g) and blend for a few seconds to mix.

5. Add sufficient potassium bicarbonate to raise the pH to approximately 4 (3.75 mL raised the pH to 4.3) and blend for a few seconds to mix.

6. Filter the fluid from the mixture by squeezing the mixture through a fine cloth. Above a cup, spoon some of the mixture onto the cloth, raise the sides of the cloth to surround the mixture, and twist the cloth to squeeze the mixture, allowing the fluid to pass through the cloth into the cup. Approximately 350 mL of liquid is obtained. The (now dry) pulverized garlic remains weigh approximately 107 g.

7. Let the fluid stand for a day at room temperature.

8. Use a food dehydrator (trays with hot air blown across them—normally used for drying fruit, vegetables, or beef jerky) to dry the fluid (I used an Excalibur). I used multiple 18-well silicone nonstick donut baking molds (by Freshware) to hold the fluid, with ½ teaspoon of fluid in each well (approximately 140 wells total). Drying time is approximately 24 hours at 95 degrees F., followed by 24 hours at 155 degrees F.

9. Remove the “crystallized” product from the wells of the molds (approximately 83 g total).

10. Grind the crystallized product using a hand cheese grater (I used a Zyliss) to make a powder.

11. Fill size 00 capsules with the powder (I used the Cap-M-Quik manual capsule filler). Makes 115 capsules, each with an estimated 35 mg of SAMNAC (11 mg of allyl mercapto groups) and an estimated 75 mg of “excess” N-acetylcysteine.

I have taken these capsules at 6 per day for a month with no noticeable side effects.

### 8.2 An “Onion” Dietary Supplement Capsule

Just as crushing garlic produces the thiosulfinate allicin (diallyl thiosulfinate), crushing onions produces dipropenyl thiosulfinate (QK475.A43B56:150), although yellow onions only have approximately 10% of the thiosulfinate yield from garlic (approximately 0.4 mg/g). Dipropenyl thiosulfinate contains propenyl mercapto groups (analogous to the allyl mercapto groups of allicin) and has the same reaction properties. Therefore, the reaction product S-propenyl-N-acetylcysteine (analogous to S-allyl-N-acetylcysteine, SAMNAC) rapidly can be formed from the mixture of dipropenyl thiosulfinate with N-acetylcysteine at a pH of approximately 4.

A dietary supplement capsule containing S-propenyl-N-acetylcysteine was produced by the following method:

1. Start with two large fresh yellow onions (637g, which should produce approximately 250 mg of dipropenyl thiosulfinate when pulverized).

2. Grate the onion with a food processor (I used a KitchenAid food processor).

3. Transfer the “mash” to a blender (I used an Osterizer), add 200 mL of water, and blend for 10 minutes to completely pulverize the onion.

4. Add 15 mL of N-acetylcysteine powder (11.5 g) and blend for a few seconds to mix.

5. Add sufficient potassium bicarbonate to raise the pH to approximately 4 (3.75 mL raised the pH to 4.5) and blend for a few seconds to mix.

6. Filter the fluid from the mixture by squeezing the mixture through a fine cloth. Above a cup, spoon some of the mixture onto the cloth, raise the sides of the cloth to surround the mixture, and twist the cloth to squeeze the mixture, allowing the fluid to pass through the cloth into the cup. Approximately 600 mL of liquid is obtained. The (now dry) pulverized onion remains weigh approximately 180 g.

7. Let the fluid stand for a day at room temperature.

8. Use a food dehydrator (trays with hot air blown across them—normally used for drying fruit, vegetables, or beef jerky) to dry the fluid (I used an Excalibur). I used multiple 18-well silicone nonstick donut baking molds (by Freshware) to hold the fluid, with one teaspoon of fluid in each well (approximately 120 wells total). Drying time is approximately 24 hours at 95 degrees F., followed by 24 hours at 155 degrees F.

9. Remove the “crystallized” product from the wells of the molds (approximately 47 g total).

10. Grind the crystallized product using a hand cheese grater (I used a Zyliss) to make a powder.

11. Fill size 0 capsules with the powder (I used the Cap-M-Quik manual capsule filler). Makes 90 capsules, each with an estimated 8.5 mg of S-propenylmercapto-N-acetylcysteine (2.7 mg of propenyl mercapto groups) and an estimated 120 mg of “excess” N-acetylcysteine.

I have taken these capsules at 6 per day for a week with no noticeable side effects.

### 8.3 A Nutraceutical Food (Garlicky Potatoes with Bacon)

Garlicky potatoes with bacon (a nutraceutical food) was prepared in two stages. First a “SAMNAC garlic powder” was produced, which was then used in the preparation of the food.

To make the SAMNAC garlic powder:

1. Start with 210 mL of garlic powder (100 g), which should produce approximately 290 mg of allicin when mixed with water, given that the average allicin yield from garlic powder is 2.9 mg/g (RM666.G15K6313:93).

2. Add 15 mL of N-acetylcysteine powder (11.5 g) and blend for a few seconds to mix.

3. Add sufficient potassium bicarbonate to raise the pH to approximately 4 (3.75 mL raised the pH to 4.5) and blend for a few seconds to mix.

4. Let the mixture stand for a day at room temperature.

8. Use a food dehydrator (trays with hot air blown across them—normally used for drying fruit, vegetables, or beef jerky) to dry the fluid (I used an Excalibur). I used multiple 18-well silicone nonstick donut baking molds (by Freshware) to hold the fluid, with one teaspoon of fluid in each well (approximately 65 wells total). Drying time is approximately 24 hours at 95 degrees F., followed by 36 hours at 155 degrees F.

5. Remove the “crystallized” product from the wells of the molds (approximately 102 g total).

6. Grind the crystallized product using a hand cheese grater (I used a Zyliss) to make a powder.

11. Each gram of the powder has an estimated 9.2 mg of SAMNAC (2.9 mg of allyl mercapto groups) and an estimated 110 mg of “excess” N-acetylcysteine.

To make the garlicky potatoes with bacon:

1. Cut three medium size russet potatoes into small cubes, approximately 1 cm on each side.

2. Put the cut potatoes in a pot with enough water to cover them.

3. Add one teaspoon (5 ml, 3 g) of the SAMNAC garlic powder to the pot.

4. Heat the pot to a gentle boil and cook until the potato cubes are done.

5. Cut 5 slices of bacon into pieces of approximately 1 cm by the width of the bacon slice.

6. Fry the bacon at 350 degrees F. until done.

7. Drain the potatoes.

8. Add the potatoes to the bacon in the frying pan.

9. Mix and fry until the excess water on the surface of the potato cubes is gone.

10. Serves two. The potatoes have a strong bacon flavor and a moderate garlic flavor. Each serving has up to an estimated 13 mg of SAMNAC and up to an estimated 165 mg of N-acetylcysteine.

### 8.4 A nutraceutical Beverage

Garlicky tomato juice can be made by:

1. To 8 ounces of tomato juice, add one teaspoon (5 ml, 3 g) of SAMNAC garlic powder.

2. Refrigerate overnight.

3. The garlicky tomato juice has a strong tomato flavor and a moderate garlic flavor. Each serving has an estimated 27 mg of SAMNAC and an estimated 230 mg of N-acetylcysteine.

### 8.5 Experiments with Other Starting Ingredients

These partially successful experiments were performed at home using “kitchen chemistry”. Although it is not necessary for patentability to have developed a commercial scale manufacturing process (and the methods described above are sufficient for patentability), the disclosure in this section illustrates some other starting ingredients that could be used for this purpose.

8.5.1 Starting with Mercaptans

Allyl mercaptan (or propyl mercaptan, 1-propenyl mercaptan, etc.) and N-acetylcysteine could be used themselves produce the mixed disulfide between allyl mercaptan and N-acetyl cysteine (SAMNAC). This would require the use of an oxidizing agent to induce disulfide formation. One traditional means for doing this is by bubbling air through the reaction mixture.

Allyl mercaptan is commercially available (including food grade, which is intended for human consumption in foods) and I have used it for experiments in the past. But even a small concentration in the air is objectionable, as was evidenced in the past by the appearance of a mysterious strong odor in Manhattan on Jan. 8, 2007 that nauseated many people. Even a few well dispersed ounces of (for example) ethyl mercaptan could be enough to stink up all of Manhattan (NYTIMES2001:0121A). Although the source of the odor was not was not found, it was described as having a mercaptan-like smell and may have been a natural product produced by micro-organisms in the coastal marshes (NYTIMES2001:0121B).

Even the thought experiment of using allyl mercaptan in a bubbling mixture was enough to prevent actually doing this (in consideration of my wife and neighbors).

But for designing an industrial production process, there should be known oxidizing agents that can be used to induce disulfide formation (hydrogen peroxide comes to mind). But for my actual experiments, there is no advantage to starting with a mercaptan instead of starting with a thiosulfinate (as described in the previous sections) or a disulfide (as described below).

Note: Allyl mercaptan is slightly dissolvable in water (5 g/L), but dissolves freely in oil, indicating that it is a lipophilic molecule. In experiments, it tends to float on top or cling to glassware rather than staying within an aqueous solution.

Although allyl mercaptan (and diallyl disulfide) have low solubility in water (and high solubility in lipids), experiments have shown that their concentrations in both the membranes and the cytosol of cells in sufficient to provide biologically significant activity (US2005/0260250A1).

8.5.2 Starting with Disulfides

An initial attempt to make SAMNAC powder starting with diallyl disulfide (DADS) and N-acetylcysteine was unsuccessful, as described in this section.

A mixture of DADS and N-acetylcysteine in water produces a solution with a pH near 2, which is much to acidic for thiol-disulfide reactions to occur. An acceptable reaction rate is obtained at a pH of 7.

The low pKa of N-acetylcysteine is due to the carboxyl group (COOH), which is normally ionized (COO⁻) at any pH over 2. For normal cysteine, the ionized carboxyl group is “cancelled out” by its ionized amino group (NH³⁺) to produce a net pH near 7 when dissolved, but in N-acetylcysteine, this amino group is acetylated so its ionization is unavailable.

By adding some potassium bicarbonate to the mixture (which reacts with H⁺ to produce H₂O and K⁺ the pH can be adjusted to approximately 7. (A target pH of 7 was desired because at this pH the amount of K⁺ will be equal to the amount of SAMNAC⁻ that is formed, which will simplify the formation of a 1:1 salt when the mixture is later dried.)

Note that the formed SAMNAC will be an anion in solution (the carboxyl group will remain ionized) and will form a salt with the added potassium when dried (unless the potassium is removed before drying). In this case, it is probably beneficial for the potassium to remain in the product, because potassium is safe and the potassium consumption of most people is lower than the current recommendations.

In the initial mixture, the DADS floats on the surface, but as it combines with the N-acetylcysteine, the surface layer disappears. Mixing occasionally speeds up this process.

Because the ratio of thiols to disulfides is not changed from thiol-disulfide exchange reactions, starting with a 10× excess of N-acetylcysteine over the DADS concentration could be expected to produce approximately 90% retained N-acetylcysteine, 9% SAMNAC, 1% allyl mercaptan and 0.1% retained DADS (the same 10× ratio of thiols to disulfides). In practice the observation that the surface layer disappears implies that essentially all of the allyl mercaptan and DADS had combined with the N-acetylcysteine. This could be explained by the relatively high solubility of SAMNAC in water (SAMNAC molecules that are formed preferentially remain as SAMNAC rather than forming insoluble allyl mercaptan or DADS in subsequent exchange reactions, resulting in an equilibrium which naturally tends to exclude these insoluble products). The implication of this is that the high level of excess N-acetylcysteine that was used probably wasn't necessary.

After the DADS has dissolved, the mixture can be dried and then ground into a powder.

Subsequent HPLC analysis performed as work for hire by Dr. Lawson showed that the powder did not contain a significant amount SAMNAC.

An experiment by Dr. Lawson showed the reason why. As shown in FIG. 8, the SAMNAC concentration peaks at one hour (at over 90% of the theoretical maximum yield) but then declines.

In hindsight, the molecular structure of allyl mercaptan can explain this. The initial thiol-disulfide exchange reactions are rapid and complete within an hour. But there is still approximately 10× excess N-acetylcysteine in the mixture. The retained C═C double bond from the allyl mercaptan is vulnerable to a Michael addition reaction which could proceed to modify the SAMNAC molecules, decreasing the SAMNAC yield as shown.

Dr. Lawson's main conclusions were:
1. Under the above conditions, NAC and DADS rapidly react to form expected (near quantitative) amounts of SAMNAC, especially if the pH is around 5-6.
2. The instability of the formed SAMNAC can probably be corrected by lowering the pH, after SAMNAC formation to about 4-5.

Note: An easy way to lower the pH would be to use less N-acetylcysteine as a starting material (e.g. 2× excess instead of 10×), balance the pH for this amount of anion (which would take only ⅕ as much cation to do), and to then add N-acetylcysteine at the appropriate time (e.g. after one hour).

A subsequent experiment using dipropyl disulfide (which is similar to DADS but doesn't has a C—C single bond instead of the C═C double bond of DADS) was only partially successful. The resulting mixture was slow to dry, and after drying soon became soft and sticky (presumably due to moisture absorption from air). Before grinding could be completed, the grinder became stuck. Also, the pile of “ground” powder “melted” together too soon to allow it to be put into capsules. So the S-propylmercapto-N-acetylcysteine salt that I made is not practical for use in tablet form, but it probably could be used in soft-gel capsule form.

In hindsight, this may be due to the known fact that potassium salts are “highly hygroscopic” and readily absorb moisture from the atmosphere (RS403.H33:393).

8.5.3 Another Dietary Supplement Capsule

By using calcium carbonate to adjust the pH (instead of potassium bicarbonate), a calcium salt is formed (instead of a potassium salt), which easily forms a solid which can be ground into a powder suitable for use in a capsule.

The procedure I used is:

1. Mix 250 mL (177 g) of N-acetylcysteine and 80 mL (19.5 g) of calcium carbonate dissolved in one liter of water (the resulting pH is approximately 5.8).

2. Add 80 mL (78 g) of onion oil (a “natural” source of dipropyl disulfide, containing approximately 37% dipropyl disulfide by weight).

3. Mix for 24 hours (in air)

4. Use a food dehydrator (trays with hot air blown across them—normally used for drying fruit, vegetables, or beef jerky) to dry the fluid (I used an Excalibur). I used multiple 18-well silicone nonstick donut baking molds (by Freshware) to hold the mixture, with one tablespoon of fluid in each well (approximately 144 wells total). Drying time is approximately 24 hours at 95 degrees F., followed by 36 hours at 155 degrees F.

5. Remove the hardened product from the wells of the molds (approximately 345 g total).

6. Grind the hardened product using a hand cheese grater (I used a Zyliss) to make a powder.

7. Use a capsule filler to fill approximately 1000 size “0” capsules (I used Cap-M-Quik).

Each capsule has an estimated maximum of 63 mg of S-propylmercapto-N-acetylcysteine (20 mg of propyl mercapto groups) and an estimated 280 mg of “excess” N-acetylcysteine.

These examples are illustrative and the invention is not intended to be limited to these examples.

### 9 DOSAGES

The anticipated viable dosage range for these compositions is from a minimum of 1 mg to a maximum of 5 g. The lower end of this range encompasses unit dosages (e.g. in nutraceuticals) of which multiple servings (e.g. multiple types of these nutraceuticals) could be consumed each day (e.g. for an accumulated dosage of over 5 mg/day).

In general, the most preferred dosage range is from 5 mg to 60 mg, with a dosage rang of 1 mg to 60 mg being less preferred, and a dosage range of 20 mg to 5 g also being less preferred.

Experience with similar mercaptans has shown a dose-dependent effectiveness range of, for example, a total of 30 mg to 60 mg per day (e.g. for the treatment of chronic arsenicosis, as disclosed in U.S. Pat. No. 7,678,833). For this example, the unit dose (per capsule) was 10 mg, so the daily consumption was 3 to 6 capsules. Because arsenicosis is an extreme case, most other conditions are likely be responsive to daily dosages well below this.

The upper limit of the range is based on the dosages of mercaptans (or disulfides that metabolize to form mercaptans) that have been used in animal studies. For example, in a study of the prevention of acetaminophen poisoning in mice (PHYRES3:50), the dosage of S-allylmercaptocysteine used was up to 200 mg/kg, which would correspond to a dosage of 5,000 mg of allyl mercaptan for a 70 kg adult human. Because acetaminophen poisoning is the largest cause of poisoning from legal drugs in the US (BMCCC6:155), a dosage as large as 5,000 mg could be justified, if it is proven to be safe and effective in humans.

### 10 OTHER CLAIMED COMPOSITIONS

The examples above have emphasized the use of mercaptans which are present naturally in foods such as garlic, onions, and cabbage or are produced from these during normal food preparation and consumption. In particular, both allyl mercaptan and propyl mercaptan are already approved as food ingredients by the FDA. In addition, garlic oil, diallyl disulfide, onion oil, and dipropyl disulfide (which are all FDA approved for use in foods) metabolize during digestion to form allyl mercaptan or propyl mercaptan. In addition to their natural consumption by populations for millennia, these compounds have been used experimentally without apparent toxicity in animals at dosages up to 100 mg/kg, which would correspond to a dosage of 7,000 mg for a 70 kg adult human.

But various other organosulfur compounds can form mercaptans upon digestion, which in turn can form disulfides and antimicrobial thiosulfinates when oxidized within the body. Just as the compound diallyl disulfide can be oxygenated to form allicin, the onion-derived compounds dipropyl disulfide and n-propyl allyl disulfide can be oxygenated to their corresponding thiosulfinates, which may explain their antibiotic effectiveness against Salmonella typhimurium and E. coli (AM17:903).

Interestingly, when the essential oil is extracted from onion, it contains dipropyl groups rather than the di-1-propenyl groups that are formed when an onion is crushed (QK475.A43B56:144). Both are suitable for use for this invention, but dipropyl disulfide is the one that is currently listed as GRAS (generally recognized as safe) for use as a food additive by the FDA.

Similarly, the organosulfur compounds derived from cabbage tend to contain methyl groups, with methyl methanethiosulfinate (MMTSO) showing remarkable antimicrobial properties (JFP60:67).

A study of the nematicidal activity of various sulfur compounds from the plants “Allium grayi Regel” and “Allium fistulosum L. var. caespiitosum” concluded that those which have a disulfide, trisulfide, thiosulfinate, or thiosulfonate group are potential nematicides and antimicrobials (ABC52:2383). The most effective compound found was the thiosulfinate CH₃(CH₂)₂SSO(CH₂)₂CH₃ (dipropyl thiosulfinate).

In general, it is expected that mercapto radicals containing up to 5 carbon atoms in a linear or branched configuration, when disulfide bounded to cysteine (or a cysteine derivative), share many of the properties that are attributed to SAMC (and its derivatives). Studies of radioprotective substances have shown that mercaptans with more than 5 carbon atoms become less effective in protecting animals from radiation exposure. The explanation advanced here is that when mercaptans that are larger than this are consumed, they eventually form mixed disulfides with glutathione which are excreted from cells by “GS-X” multidrug resistance transport proteins (QP606.G59G59:199).

For the present invention, the preferred mercaptans radicals are organosulfur compounds with a carbon backbone length of three carbon atoms (e.g. the allyl mercapto radical, the propyl mercapto radical, and the 1-propenyl mercapto radical), with carbon backbone lengths of 2 or 4 being less preferred, and carbon backbone lengths of 1 or 5 being still less preferred.

SAMC has the advantage that it metabolizes to allyl mercaptan and other known derivatives of garlic that have been successfully consumed by millions of people. And its related organosulfur compounds (such as allicin) have been extensively investigated. SAMNAC has the further advantage that N-acetylcysteine is considered to be a less toxic form of cysteine, and is used clinically at much larger dosages.

The present invention, however, applies also to the more general class of compounds that have been presented in this section, which are referred to generally within this specification as “allium-related organosulfur” compounds, and are more precisely defined within this section and the claims.

Novel preparations of the invention may be made by a number of conventional methods. Nutritional wafers are within the scope of the invention. Compositions for oral dosage can include inactive components which provide for easier or more pleasant oral administration. Oral compositions may also include other active ingredients.

Methods of administration include non-oral means such as topical ointments, nasal, sublingual, intravenous and parenteral or any other method that will present the active metabolites or their prodrugs to the cellular environment of the host. The host may be any type of animal with an immune system similar to that of a mammal.

### 11 COMPARISON WITH AGED GARLIC EXTRACT (AGE)

Previously, the only commercially available dietary supplement containing SAMC was Aged Garlic Extract from Kyolic Research. The “AGE” product contains primarily the water-soluble compounds S-allylcysteine (SAC, 0.62 mg/g) and SAMC (assayed at 0.14 mg/g), but it also contains lesser amounts of the lipid-soluble compounds diallyl sulfide, triallyl sulfide, and DADS. Thus, even though it contains SAMC and other compounds, it predominantly consists of SAC. AGE contains no allicin (ISBN0683181475, page 104).

SAC contains a cysteinal radical bonded to an allyl group, with just one sulfur atom in the molecule. In contrast, SAMC contains a cysteinal radical disulfide bonded to an allyl-mercapto group, resulting in two sulfur atoms in the molecule (bonded together by the disulfide bond). Therefore SAMC can participate in disulfide exchange reactions, but SAC cannot. Also, via thiol-disulfide exchange reactions, an SAMC molecule can ultimately yield both a cysteine molecule and an allyl mercaptan molecule. In general, SAMC has more beneficial medicinal properties than SAC does. (But SAC is not ineffective, in part because it is a potent inducer of detoxification enzymes.)

The SAMC content of available dietary supplements is quite low compared to the range of concentrations that is provided by the present invention. The SAMC content of AGE is advertised as containing 200 mcg/g (somewhat above its assayed amount), and AGE-containing dietary supplements typically contain less than 1000 mg of AGE, resulting in a maximum dosage of at most 200 micrograms of SAMC per capsule. The present invention promotes dosages at least in the milligram range, typically on the order of up to 100 milligrams of SAMC per capsule, and can support dosages significantly beyond this, if such dosages are found to be beneficial. For example, SAMC dosages of up to 200 mg/kg have been used successfully in mice (EJP433:177), which corresponds to a dosage of 14,000 mg for a person weighing 70 kg.

It is interesting to note that the manufacturer of AGE (Kyoloc Research, Wakunaga Nutritional Supplements) distinguishes AGE from other garlic supplements that produce allicin (e.g. tablets that contain garlic powder), claiming that AGE does not disrupt intestinal bacteria the way that products that produce allicin do. Kyolic Research makes no claims that AGE is antimicrobial, and actually distinguishes their product for garlic products that are antimicrobial. Kyolic Research does not promote AGE (or the SAMC within it) as a broad spectrum antimicrobial agent, and seems to be unaware that SAMC could be use for this purpose.

### 12 SUMMARY

The present invention has been illustrated according to its application in the prophylactic prevention and therapeutic treatment of infectious diseases and for the prophylactic prevention and therapeutic treatment of pathologenic immune system response, but given the benefit of this disclosure those skilled in the art will realize that it can also be applied to other conditions involving inflammation, such as chronic asthma. Therefore, the invention is not to be limited to the above description and illustrations, but is defined by the appended claims.

What is claimed is:

1. A method of treating microbial infections which increases the broad spectrum antimicrobial activity localized to the environment adjacent to activated immune system cells of a mammalian host currently emitting reactive oxygen species, comprising:
administering to a mammal in need of said antimicrobial activity an effective amount of a composition selected from the group of a dietary supplement, a nutraceutical, or a drug, said composition being comprised of one or more active ingredients selected from the group of a mercaptan comprising one sulfur atom and up to 5 carbon atoms in a linear or branched configuration, or said mercaptan disulfide bonded to cysteine, or said mercaptan disulfide bonded to the cysteine of N-acetylcysteine;
wherein said composition metabolizes within said host to form said mercaptan;
wherein said mercaptan serves as an antioxidant within said host, the successive oxidation of which forming a thiosulfinate or mixed thiosulfinate, resulting is an localized thiosulfinate or mixed thiosulfinate being non-enzymatically formed in response to the localized generation of reactive oxygen species by adjacent activated immune system cells;
wherein said thiosulfinate or mixed thiosulfinate is a broad spectrum antimicrobial agent;
whereby to antimicrobial activity of the immune system cells that are currently emitting reactive oxygen species is increased by the localized formation of said broad spectrum antimicrobial agent.

2. The method of claim 1 where said mercaptan is allyl mercaptan.

3. The method of claim 1 where said mercaptan is propyl mercaptan.

4. The method of claim 1 where said mercaptan is 1-propenyl mercaptan.

5. The method of claim 1 where said mercaptan disulfide bonded to cysteine is S-allylmercaptocysteine.

6. The method of claim 1 where said mercaptan disulfide bonded to the cysteine of N-acetylcysteine is S-allylmercapto-N-acetylcysteine.

7. The method of claim 1 where said thiosulfinate is allicin.

8. The method of claim 1 where said nutraceutical contains said active ingredients within the range of 1 milligram to 1000 milligrams.

9. The method of claim 1 where said dietary supplement contains said active ingredients within the range of 1 milligram to 1000 milligrams.

10. The method of claim 1 where said drug contains said active ingredients within the range of 1 milligram to 14 grams.

11. The method of claim 1 where said microbial infection can cause pneumonia.

12. The method of claim 1 where the treatment is prophylactic treatment.

13. A method of treating inflammatory respiratory distress, comprising:
administering to a mammal in need of said treatment an effective amount of a composition selected from the group of a dietary supplement, a nutraceutical, or a drug, said composition being comprised of one or more active ingredients selected from the group of a mercaptan comprising one sulfur atom and up to 5 carbon atoms in a linear or branched configuration, or said mercaptan disulfide bonded to cysteine, or said mercaptan disulfide bonded to the cysteine of N-acetylcysteine;
wherein said composition metabolizes within said host to form said mercaptan;
wherein said mercaptan serves as an antioxidant within said host, suppressing inflammation without compromising immune system activity.

14. The method of claim 13 where said inflammatory respiratory distress is Acute Respiratory Distress Syndrome (ARDS).
